(19) United States
(12) Patent Application Publication
(10) Pub. No.: US 2004/0033273 A1
(43) Pub. Date: Feb. 19, 2004

(54) Withasol and methods of use

(75) Inventors: Bhushan Patwardhan, Pune (IN); Aravind H. Kapadi, Pune (IN)

Correspondence Address: Pillsbury Winthrop LLP, Suite 1800, 101 W. Broadway, San Diego, CA, 92101, US

(73) Assignee: AyurCore, Inc., San Jose, CA

(21) Appl. No.: 10/074,146

(22) Filed: Jan. 11, 2002

Related U.S. Application Data
(60) Provisional application No. 60/269,214, filed on Feb. 14, 2001.

(51) Int. Cl. A61K 35/78; A01K 67/00

(52) U.S. Cl. 424/725; 800/10

(57) ABSTRACT

The present invention relates to compositions useful for ameliorating or reversing naturally occurring immunosuppression or myelosuppresive, or side effects of myelosuppresive or immunosuppresive drug therapy. Medicinal fractions derived from the plant Withania Somnifera that reverse, at least in part, one or more characteristics of immunosuppression or myelosuppression and a process for manufacturing the fractions are particular aspects of the invention. Withania Somnifera medicinal fractions have additional biological activities including anti-tumor potentiating activity.

### RELATED APPLICATIONS

This application claims the benefit of priority of application serial No. 60/269,214, filed Feb. 14, 2001.

### TECHNICAL FIELD

This invention relates to a composition obtained from a plant that has immune stimulating activity or anti-tumor activity, and more particularly to Withania Somnifera fractions and mixtures of fractions having one or more of these activities.

### BACKGROUND

The immune system plays an important role in response to infection and disease and is among the most rapidly developing area of biomedical research for the prevention and treatment of a wide range of disorders. Arthritis, ulcerative colitis, asthma, allergic reactions, parasitic and infectious diseases are now primarily considered to be immunological disorders. Immune mechanisms are also involved in a variety of other diseases such as diabetes mellitus, cancer, myocardial diseases, cirrhosis and atherosclerosis.

Immunostimulation and immunosuppression can regulate normal immunological functioning. Suppression or deficiency of immune function result from hypoactivity whereas inflammatory and allergic manifestations are thought to be the direct cause of hyperactivity of immune function in non-specific immunity. Non-specific immunity is mediated, inter alia, by granulocytes, macrophages, natural killer cells, complement and properdin, as well as effector substances, which include interleukins, tumor necrosis factor, interferons, lysozymes, prostaglandins, oxygen radicals and other mediators. The function and efficiency of non-specific immunity may be influenced by exogenous and endogeneous factors such as physical and psychological stress, hormonal imbalance, pharmaceuticals and the like. Hence, modulating immune response by treating with immunostimulating or immunosuppressing agents can alleviate disease associated with immune hypoactivity and hyperactivity, respectively.

Most of the chemical agents known to affect the immune system are immunosuppressants and cytotoxic agents. For example, cyclosporin A is used in organ transplantation, azathioprine inhibits DNA synthesis, cyclophosphamide is relatively selective for lymphoid tissue, thiocarbomate has direct cytotoxic effects. Many chemotherapeutic agents available today are also immunosuppressants. There are however few immunostimulating agents available.

Plants having pharmacological and biological activity have been the basis of treatment of human diseases from time immemorial. Every country in the world has herbal remedies for the treatment of diseases and various human conditions. The foundation of the modem drug industry is practically based on compounds obtained from plants that have been developed further synthetically to obtain more suitable analogues. For example, the isolation of morphine from opium poppy, Quinine from the cinchona tree, cocaine from the leaves of Coca shrubs, and a host of drugs such as atropine, curare, digoxin, reserpine, and the like.

Naturally occurring plant material often contains a series of closely related compounds produced naturally via biological and biochemical reactions. The analogues often have similar activities and, furthermore, together may exercise a synergistic effect on the pharmacological or biological activity and, at the same time, may suppress toxic side effects. However, a drawback in using plant material in its crude form is that the dosages required of such material, to be therapeutically beneficial, are quite high, sometimes up to 10 gms a day or more.

In India, Ayurveda has carried out studies for many generations and recorded medicinal uses of plants for over 5000 years. Withania Somnifera (Solanaceae) plant is native to the Indian sub-continent and has been studied for its chemistry, pharmacology and clinical efficacy (K. Sharma et al., Indian Drugs, 29(6):247-255 (1992)). Chemical constituents of Withania Somnifera include alkaloids (withanine, withasomnin) and steroidal glycosides (sitoindosides and withanolides; D. Lavie, Phytochemistry, 14:189 (1975)). Sitoindosides and Withanolides are suspected of being responsible for particular biological activities of Withania Somnifera, such as antiarthritic and antirheumatic activity (K. Anabalgen et al., Indian Journal of Experimental Biology, 19:245-249 (1981); N. P. Bactor et al., Journal of Research in Indian Medicine, 5(2):72 (1971)).

Several compounds in Withania Somnifera, particularly withanolides, are thought to interact with the liver protein synthetic machinery and influence many modular proteins. A bulk of evidence indicates an apparent anti-inflammatory and anti arthritic activity of the plant against various models of inflammation in carageenan, Cotton pellet granuloma and adjuvant arthritis, where long-term administration has shown significant radiographical changes, reduction in erythrocyte sedimentation rate (ESR) and acute phase symptoms, including C-reactivity proteins (Begum and Sadique, Indian Journal of Experimental Biology, 26(11):877 (1988)). Bactor et al. studied 118 cases of arthropathies, including 78 of rheumatoid arthritis, and showed that Withania Somnifera is useful in acute rheumatoid arthritis. Chronic cases were improved and significant decrease in ESR noted. No side effects were observed during the treatment and follow-up studies up to six months for doses ranging from 4-6 g/day. Nevertheless, in spite of experimental data on the usefulness of Withania Somnifera as an analgesic, anti-inflammatory, antiarthritic, antirheumatic and immunomodulatory properties, very few systematic clinical trials have been conducted.

Non-steroidal anti-inflammatory drugs have three major actions, all of which are related to inhibition of cyclo-oxygenase resulting in decreased formation of prostanoids. Firstly, an anti-inflammatory action achieved by reduced production of vasodilator prostaglandins (PGE2, PGI2), which means less vasodilation and, indirectly, less oedema. Secondly, an analgesic effect achieved by reduced prostaglandin production (less sensitization of nociceptic nerve endings to the inflammatory mediators bradykinin and 5-hydroxytryptamine). Thirdly, an antipyretic effect that is probably due to a decrease in the mediator PGE2 generated in response to inflammatory pyrogens, much as interleukin-1. Since ginger inhibits prostanoid synthesis and also products of 5-lipoxygenase, its ameliorative effects in arthritis and muscular discomforts could be related to reduced formation of prostanoids and leukotrienes. Because of such a possibility, a decrease in the carageenan-induced oedema formation in the rat's paw after 3 g of ginger extract administration has been demonstrated and the potency of the extract in the acute inflammation test appears to be comparable to that exhibited by acetyl salicylic acid reported in the same study. (Mascolo N. et al., Journal of Ethnopharmocology, 27:129-140 (1989)).

Thus, the existence of immunosuppression due to naturally occurring phenomenon, environmental factors or the use of agents that cause defective, deficient, or aberrant immune response, directly or indirectly, creates a need for compounds that counteract immunosuppresant activity. Plant extracts of the present invention have immunomodulatory activity and other activity (e.g., anti-tumor activity) and, therefore, will benefit patients having pathological disorders, including immunosuppression or hyperplasia, as well as being useful in patients for immunopotentiation (e.g., increasing vaccination efficacy).

### SUMMARY

The present invention generally relates to compositions useful for ameliorating immunosuppression or side effects of myelosuppressive or immunosuppresive drug therapy. Particularly, this invention relates to medicinal fractions and a process for manufacturing the fractions derived from the plant Withania Somnifera. The fraction(s) have immunostimulating activity in animals. The fractions additionally have anti-tumor activity in animals, in particular, when used in combination with other anti-tumor drugs.

Thus, in accordance with the invention, there are provided compositions having immune stimulating activity or anti-tumor activity obtained from Withania Somnifera, and methods of producing such compositions. In one embodiment, a composition of the invention is prepared by contacting Withania Somnifera plant or plant part (e.g., root, stem leaf, seed, seedling, etc.) with a first medium polar solvent (e.g., acetone, tetrahydrofuran or ethylacetate or an alcoholic organic solvent) to produce a particulate suspension; clarifying the particulate suspension to produce a clarified first solution and a first residue; evaporating the solvent from the first clarified solution to produce a fraction, denoted fraction A; resuspending the first residue in a second polar solvent (e.g., about 50% ethanol or about 40 to 60% isopropyl alcohol, a mixture of water and isopropyl alcohol (IPA), or an alcoholic organic solvent) thereby producing a second solution and a second residue; clarifying the second solution to produce a second clarified solution; evaporating the second polar solvent from the second clarified solution to produce a fraction, denoted fraction B; resuspending the second residue in a third solvent (e.g., water) more polar than the second polar solvent thereby producing a third solution and a third residue; clarifying the third solution to produce a third clarified solution; evaporating the third solvent from the third clarified solution to produce a fraction, denoted fraction C; combining fractions A, B and C to produce an extract; resuspending the extract in a solution to produce a fourth alkaline solution; and fractionating the fourth solution with a non polar solvent (e.g., methylene chloride, diethyl ether or chloroform) and removing the solvent to produce a composition having immune stimulating activity or anti-tumor activity (e.g., having one or more alkaloids or withanolides). In one aspect, fractions A, B and C are combined in approximately equal proportions by mass. In another aspect, fractions A, B and C are combined in unequal proportions by mass. In yet another aspect, the plant or plant part is soaked in the first solvent for at least about 2 hours.

A composition having immune stimulating activity or anti-tumor activity can be characterized as having a TLC profile the same or substantially the same as a profile set forth in FIGS. 1A, 2A or 3A, the profile obtained with a hexane:methylene chloride:methanol mobile phase in about a 20:30:2 ratio. The composition can also be characterized as having an HPLC profile the same or substantially the same as a profile set forth in FIGS. 1B, 2B or 3B, said profile obtained using a reverse-phase C-18 column at a flow rate of about 1.2 ml/min with a mobile phase of methanol:water in a ratio of about 60:40.

A composition having immune stimulating activity or anti-tumor activity can also be characterized by its physical properties. In one embodiment, a composition obtained from Withania Sommifera having immune stimulating activity or anti-tumor activity is soluble in water; substantially free of alkaloids; and has at least one glycowithanolide. Exemplary glycowithanolides that may account, at least in part, for immune stimulating activity or anti-tumor activity include sitoindoside IX, sitoindoside X, a mixture thereof, or one or more glycowithanolides distinct from sitoindoside IX or sitoindoside X (e.g., two, three, four, etc., or more glycowithanolides distinct from sitoindoside IX or sitoindoside X).

In another embodiment, a composition obtained from Withania Somnifera having immune stimulating activity or anti-tumor activity is substantially free of withanolides. In yet another embodiment, a composition obtained from Withania Somnifera having immune stimulating activity or anti-tumor activity has a glycowithanolide content from about 0.5 to 1.6% by weight. In still another embodiment, a composition obtained from Withania Somnifera having immune stimulating activity or anti-tumor activity has one or more of the following by mass: about 35-75% protein content; about 0.5 to about 5% glycowithanolide(s); about 3 to about 10% ash; or about 30 to about 60% carbohydrate.

Compositions may be further fractionated in order to obtain purer forms. For example, immune stimulating activity or anti-tumor activity may include one or more of the molecules in peaks 1 to 5 or 7 to 9 set forth in FIGS. 1A, 2A or 3A, or a combination of two or more molecules in said peaks. Thus, the invention also provides further fractionated or purified compositions, such as compositions including one or more of the molecules in peaks 1 to 5 or 7 to 9 set forth in FIGS. 1A, 2A or 3A, or a combination thereof.

Compositions of the invention have immune stimulating activity in vivo. In one embodiment, administering about 50 mg/kg subject mass of the composition to a Balb-c mouse increases by about 20% or more the number of white blood cells in the Balb-c mouse. In another embodiment, administering about 100 mg/kg subject mass of the composition to a Balb-c mouse increases by about 20% or more the number of white blood cells in the Balb-c mouse. In various aspects, the compositions are active in subjects having normal or less than normal numbers of white blood cells.

Compositions having immune stimulating activity or anti-tumor activity may be formulated for in vivo administration. Such formulations may further be formulated for systemic or targeted administration, such as formulations compatible with injection or infusion to target tissues or organs, or oral administration such as pills, capsules, syrups, or suspensions.

Thus, in another embodiment, the invention provides pharmaceutical formulations including invention compositions and pharmaceutically acceptable carriers. Such formulations may be combined with other drugs in order to provide an additive or synergistic effect. Thus, in one aspect, an invention composition and pharmaceutically acceptable carrier further includes a drug, such as a drug that increases white blood cell numbers, has immunosuppressing activity, has anti-cell proliferative activity (e.g., anti-tumor activity), or inhibits cell proliferation or cell cycle progression in a subject. Exemplary drugs having anti-cell proliferative activity and, hence anti-tumor activity that may be included with the compositions of the invention are drugs that inhibit nucleic acid or protein synthesis, steroid glycosides, alkylating agents, anti-metabolites, plant alkaloids, plant extracts, antibiotics, nitrosoureas, hormones, nucleoside analogues, or nucleotide analogues. Specific examples include, for example, cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-AZC, taxol, vinblastine, vincristine, doxorubicin, bleomycin, actinomycin D, mithramycin, mitomycin C, carmustine, lomustine, semustine, streptozotocin, hydroxyurea, cisplatin, mitotane, procarbazine, dacarbazine and dibromomannitol.

Invention compositions may be further packaged into kits, the kits optionally containing instructions for use. In one embodiment, a kit includes an invention composition (e.g., a pharmaceutical formulation), and instructions for use in stimulating an immune response. In another embodiment, a kit includes an invention composition (e.g., a pharmaceutical formulation), and instructions for use in stimulating anti-cell proliferative activity, for example, in potentiating an anti-cell proliferative therapy.

As the invention compositions have one or more of immune stimulating or anti-cell proliferative activities, the invention also provides methods for stimulating an immune response (e.g., increasing the number of white blood cells) or anti-cell proliferative activity in a subject. In one embodiment, a method includes administering to a subject an amount of an invention composition effective to increase the number of white blood cells in the subject. In one aspect, the white blood cells are selected from monocytes, macrophages, natural killer cells, dendritic cells, granulocytes, basophils and eosinophils. In another aspect, the subject has less than normal numbers of white blood cells.

Subjects in which the compositions may be administered and methods practiced include immunosuppressed subjects or subjects at risk of immunosuppressive. Examples of such subjects are those undergoing immunosuppressive therapy or will be undergoing an immunosuppressive therapy. Specific examples of immunosuppressive therapies include treatment for asthma, rheumatoid arthritis, or psoriasis.

Thus, the invention also provides methods for reducing immunosuppression in a subject. In one embodiment, a method includes administering to an immunosuppressed subject, or a subject at risk of immunosuppression (e.g., prohylactically), an amount of an invention composition effective to reduce immunosuppression in the subject.

For immunosuppression, amounts administered are those effective to inhibit immunosuppression or at least prevent further immunosuppression, as assessed by increasing or stabilizing blood cell counts, antibody titters, etc. Exemplary amounts administered comprise a dose of about 10 to 50, 50 to 100 to 200 mg composition/kg subject mass.

Subjects in which the compositions may be administered and methods practiced also include subjects undergoing an anti-cell proliferative therapy (e.g., cancer therapy) or at risk of undergoing an anti-cell proliferative therapy (e.g., prohylactically). Specific examples of cancer therapy include administration of radiation or a radioisotope. Specific examples of subjects include those at risk of, presently has or previously had cancer.

Thus, the invention further provides methods for increasing activity of an anti-cell proliferative therapy (e.g., anti-tumor drug) comprising administering to a subject treated with an anti-cell proliferative therapy (e.g., anti-tumor drug) an invention composition prior to, contemporaneously with or after treatment with the anti-cell proliferative therapy (e.g., anti-tumor drug) to the subject. In various aspects, the anti-cell proliferative therapy is an anti-tumor drug, for example, a radioisotope, an alkylating agent, an anti-metabolite, a plant alkaloid, a plant extract, an antibiotic, a nitrosourea, a hormone, a nucleoside analogue, or a nucleotide analogue.

For anti-cell proliferative activity, amounts administered are those effective to inhibit cell proliferation or growth, as assessed by a stabilization of by decreasing the numbers of cells. For example, an amount that reduces or stabilizes the size of a tumor mass is an effective amount. Exemplary amounts administered comprise a dose of about 10 to 50, 50 to 100, or 100 to 200 mg composition/kg subject mass.

Cell proliferation which may be treated by a method of the invention include benign hyperplastic disorders, such as fibrosis and scarring. Cancerous disorders (metastatic or non-metastaic) are another example of cell proliferation which may be treated in accordance with the invention. In various embodiments, a cancer comprises a solid (e.g., fibrosarcoma, lymphosarcoma, liposarcoma or osteosarcoma) or liquid (e.g., lymphoma, leukemia or myeloma) tumor. In additional embodiments, a cancer comprises a breast, brain, head or neck, eye, nasopharynx, lung, liver, pancreas, kidney, esophagus, stomach, small or large intestine, bladder, rectal, prostate, testicular, ovarian, uterine, bone, muscle or skin tumor.

In the methods stimulating an immune response or reducing immunosuppression or increasing activity of an anti-cell proliferative therapy, compositions may be administered using any protocol to achieve the desired effect. Thus, in various embodiments, the composition is administered at intermittent frequencies or variable dosages; administered in single or multiple doses; administered via injection, gradual perfusion or intubation or orally; or administered prior to, contemporaneously with, or after administering a therapy or drug (e.g. an immune stimulating therapy or an anti-immunosuppression therapy, or an anti-cell proliferative therapy such as a drug).

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B are TLC and HPLC profiles, respectively, of WITHASOL batch 1. There are a total of 9 peaks in the TLC profile, of which Withanolide-D corresponds to peak no. 6. Withanolide-D is as indicated in the HPLC chromatogram.

FIGS. 2A and 2B are TLC and HPLC profiles, respectively, of WITHASOL batch 2. Withanolide-D is indicated as above.

FIGS. 3A and 3B are TLC and HPLC profiles, respectively, of WITHASOL-batch 3. Withanolide-D is indicated as above.

FIG. 4 is a graph of the dose-activity relationship of WITHASOL.

### DETAILED DESCRIPTION

The invention is based, in part, upon a composition obtained from Withania Somnifera having immune stimulating activity or anti-tumor activity in vivo. A composition of the invention obtained from Withania Somnifera includes one or more components having immune-stimulating activity, including, for example, anti-myelosuppressive activity and increased humoral immune responsiveness. A composition of the invention obtained from Withania Somnifera also has one or more components having anti-tumor activity, particularly when used in combination with an anti-tumor agent, where it potentiates anti-tumor activity of the anti-tumor agent. Thus, the compositions of the invention are useful for treating physiological conditions associated with myclosuppression, stimulating immune response, or for use alone or as an adjunct to anti-cell proliferative (e.g., anti-cancer) therapy. As an example, a subject that is immunosuppressed (e.g., myelosuppressed) or at risk of immunosuppression can be treated with a composition of the invention in order to reverse or prevent, at least in part, one or more characteristics of immunosuppression.

Compositions of the invention have additional activities indicating applicability for treating other disorders or modulating other physiological functions. For example, compositions of the invention modulate numbers of particular cell types associated with immune-responsiveness. The composition can therefore be used to modulate numbers of particular cell lineages, types or subtypes in a subject, for example.

Thus, in accordance with the invention, there are provided compositions having immune stimulating activity or anti-tumor activity. In one embodiment, a composition is obtained from Withania Somnifera by a method of the invention. The composition can be obtained from the whole or a part of the Withania Somnifera plant, such as roots, stems, shoots, leaves, flowers, seeds, seedlings, etc. The plant can be of any age, can be dried or otherwise preserved (e.g., refrigeration) prior to preparing the composition.

The compositions of the invention are readily available as they are obtainable from a plant source, as described herein. Large quantities can be produced by scaling up the preparation protocol for economy of size. It is also to be understood that plants within the genera of Withania can be used as starting material for obtaining the compositions of the invention. Such plants include, for example, Withania aristata, frutescens and coagulans. Withania plants and plant parts further include genetically engineered variants of Withania as well as cultured Withania plant cells, organs or tissues, from any portion of the plant, including progeny thereof.

“Immunomodulation” means an alteration or change in cellular (cell-mediated) or humoral (antibody-mediated) immunity. Immunomodulation includes “immunostimulation,” “stimulating immune response,” “immune stimulating activity,” and grammatical variations thereof, which mean an enhancement or improvement of an immune response, which includes stimulation of function or efficiency of cellular or humoral immunity, e.g., lymphocytes, monocytes, granulocytes, antibody, complement and properdin, and effector substances that mediate or regulate the response including interleukins, interferons, chemokines, cytokines, lysozymes, prostaglandins, oxygen radicals, components of the complement cascade and others. Immunomodulation also includes “immunosuppression,” which means a reduction of an immune response or one or more cell types or components, (e.g., antibody titre) that are associated with immune-responsiveness.

The terms also include changes in activity or amount of one or more factors that modulate the immune response pathway but which change may or may not be manifested in a change in immune responsiveness. For example, interferons are antiviral glycoproteins produced in response to virus infections. There is evidence that interferon binds to cell surface receptors and triggers secondary intracellular changes which result in decreased susceptibility of cells to viral replication. Interferon also has an additional activity of inducing natural killer (NK) cell function and recruits NK cells from undifferentiated stem cells. Thus, there may be cases where the amount of interferon is outside of the normal range but which does not result in alteration of an immune response to a viral infection. Such alterations outside of the normal range can be detected using immunoassays or functional assays to detect the amount or activity present, using techniques known in the art.

Chronic or acute immunosuppression is characterized by one or more of the following phenomenon: Decreased amounts of lymphocytes (e.g., B cells, T cells or natural killer cells), monocytes (e.g., macrophages, antigen-presenting cells, dendritic cells, Kupffer cells, Langerhans cells, microglial cells or mesangial cells), leukocytes/granulocytes (e.g., neutrophils, basophils, cosinophils or mast cells); decreased motility (chemotaxis) towards the site of infection; deficient or defective expression of cell surface immunoglobulin; decreased or inefficient cell surface antigen processing or presentation by immune cells; decreased or inefficient antibody response or production; aberrant or decreased inflammatory response due to insufficient or absence of immune stimulatory molecules (interferons, interleukins, chemokines, cytokines, lysozymes, prostaglandins, oxygen radicals, components of the complement cascade, etc.); increased tolerance to foreign antigen exposure; increased susceptibility to opportunistic infection, etc.

A composition of the invention that decreases, inhibits, reduces or reverses immunosuppression can improve one or more characteristics of immunosuppression described herein or otherwise known in the art. For example, a composition that decreases immunosuppression may increase the number of immune cells that participate in or modulate (i.e., are associated with) the immune response, such as lymphocytes (e.g., B cells, T cells or natural killer cells), monocytes (macrophages, antigen-presenting cells, dendritic cells, Kupffer cells, Langerhans cells, microglial cells or mesangial cells), leukocytes/granulocytes (e.g., neutrophils, basophils, eosinophils or mast cells), or increase antibody production (e.g., hemagglutinating antibody titre, HA titre, and complement fixing antibody titre, hemolytic antibody (HL) titre), as described herein.

As used herein, the term “myelosuppression” means a decrease in the number of myeloid origin cell types associated with immune response. Myelosuppression includes any decrease in myeloid cell types in general, that is a decrease in a broad spectrum of myeloid origin cell types, or a particular type of myeloid cell, such as a monocyte, macrophage, or antigen-presenting cell (e.g., dendritic cell). Reversal, inhibition or prevention of myelosuppression therefore means to increase the number of one or more cell types of myeloid origin in a subject; to prevent, all or in part, a decrease in the number of one or more cell types of myeloid origin in a subject; or reverse, all or in part, a decrease in the number of one or more cell types of myeloid origin in a subject. A composition that reverses myelosuppression or has myelostimulating activity can therefore restore, at least in part, numbers of one or more cell types of myeloid origin, such as neutrophils, lymphocytes, basophils, eosinophils, monocytes, macrophages, antigen-presenting cells, dendritic cells, Kupffer cells, Langerhans cells, microglial cells or mesangial cells, etc. Myelosuppression therefore is limited to decreases in numbers of cells associated with the immune response.

As used herein, the term “aberrant” means abnormal or atypical. When used in reference to an immune response or an activity or a component of the immune response pathway, the term includes immunostimulation or immunosuppression of one or more components of the pathway in comparison to that which is considered normal for a normal matched subject (i.e., accounting for age, sex, race, geographic location, any drug therapy, environmental exposure, etc.). Typically, an aberrant immune response leads either to suppression or over-stimulation of an immune response (a CTL or antibody mediated response). As described herein, a suppressed immune response in an animal can be reversed, at least in part, by administering a composition of the invention.

Naturally occurring plant material often contains a series of closely related compounds produced naturally via biological and biochemical reactions. Many of the analogues can therefore possess similar activities and, furthermore, exercise a synergistic effect on the pharmacological or biological activity of compatible compounds. Thus, the compositions described herein are likely to contain related compounds that have similar if not synergistic biological activities.

In accordance with the present invention, there are provided methods for obtaining a composition having immune stimulating activity or anti-tumor activity. In one embodiment, a method includes contacting Withania Somnifera plant or plant part with a first medium polar solvent to produce a particulate suspension; clarifying the particulate suspension to produce a clarified first solution and a first residue; evaporating the solvent from the first clarified solution to produce a fraction, denoted fraction A; resuspending the first residue in a second polar solvent thereby producing a second solution and a second residue; clarifying the second solution to produce a second clarified solution; evaporating the second polar solvent from the second clarified solution to produce a fraction, denoted fraction B; resuspending the second residue in a third solvent more polar than the second polar solvent thereby producing a third solution and a third residue; clarifying the third solution to produce a third clarified solution; evaporating the third solvent from the third clarified solution to produce a fraction, denoted fraction C; combining fractions A, B and C to produce an extract; resuspending the extract in a solution to produce a fourth alkaline solution; and fractionating the fourth solution with a non polar solvent and removing the solvent to produce a composition having immune stimulating activity or anti-tumor activity. Thus, a composition having immune stimulating activity or anti-tumor activity produced by a method of the invention is provided.

As discussed, any part of the plant can be used for obtaining a composition. In general, the plant or part thereof is cut into large chunks, medium size pieces or smaller particulates, or can be ground to form a semisolid paste like substance. The solvent is added and mixed with the plant or plant part. In order to increase yield of the active component(s) from the plant or plant parts, the mixture of plant or plant parts and solvent can be left soaking for a period of time before clarifying. Typical soaking times range from minutes to hours (e.g., 4, 8, 12, 24 or more) or days, soaking can be brief or for longer periods of time. In one embodiment, the plant or plant part is soaked in the first solvent for at least about 2 hours. In additional embodiments, the plant or plant part is soaked in the first solvent for about 2 to 4 hours, 4 to 6 hours, 6 to 8 hours, 8 to hours, 12 to 16 hours, 12 to 24 hours, or 24 to 48 hours.

Methods of the invention can employ various types of solvents having the requisite polarity. The term “solvent” as used herein also includes mixtures of solvents in the same or differing proportions. Polarities will be appropriate for the fractionation step and solvent polarity can be indicated by an index. For example, a least polar solvent has a polarity index value of 0.00 and a most polar solvent has a polarity index value of 10.0. Hexane is an example of a least polar solvent. A medium polar solvent therefore has a polarity index value of about 5.0 to about 6.0. Acetone has a polarity index of 5.0. Water-IPA mixture has a polarity index from about 5.0 to 6.5, depending on the relative percentages of each, the greater the % of water, the greater the polarity index. Other possible solvents could be methanol (5.1), n-propanol (4.0), ethanol (5.2) mixed with water at different proportion. Quantitative rankings of solvents (“Polarity Index”) can be constructed on the basis of such properties as the refractive index, electric permittivity (also known as dielectric constant), Hildebrand's solubility parameter, the modulus of the molecular dipole moment, and various functions thereof. Polarity indices for various liquids are available in published tables (see e.g. Street and Acree, J. Liq. Chromatography, 9, 2799 (1986)).

In one embodiment, a method includes contacting Withania Somnifera plant or plant part with a first medium polar solvent. Examples of appropriate medium polar solvents include acetone, tetrahydrofuran and ethyacetate. In another embodiment, the first residue is resuspended in a second polar solvent. In on aspect, a second polar solvent comprises about 50% ethanol or about 40% to 60% isopropyl alcohol (e.g., about 50% isopropyl alcohol in water). In another embodiment, the second residue is resuspended in a third solvent more polar than the second polar solvent. In one aspect, the third solvent comprises water. In another aspect, the second solvent comprises about 50% isopropyl alcohol in water. In additional embodiments, the first or second solvent comprises water or an alcoholic organic solvent. Buffers, solutions and solvents can additionally contain salts, chelating agents, preservatives and stabilizing agents (e.g., to stabilize pH within a particular range), which can be useful for removing impurities, increasing yield, or stabilizing or preventing degradation.

The residues generated throughout the method steps can be resuspended in a compatible buffer before or after adding solvent, so long as a subsequent step, if any, produces the desired product. A desired product is that which is compatible with subsequent method steps and produces fractions A, B and C having one or more activities described herein, e.g., immune stimulating activity or anti-tumor activity. Following production of fractions A, B and C, the fraction(s) is resuspended in a solution that is made alkaline (i.e., pH 7.0 or less) prior to fractionation with a nonpolar solvent. Typically, buffers and solutions will have greater than 50% water, for example, 60%, 70%, 80%, or 90% water and will have low amounts of solvent (e.g., methylene chloride, chloroform, diethyl ether, etc.), such that the solvent does not prevent a subsequent method step.

In the methods and compositions of the invention, fractions denoted A, B and C can be combined in any proportion so long as fractionating the combination with a non polar solvent produces a composition with one or more activities described herein, for example, immune response stimulating, myelosuppression inhibiting, or anti-cell proliferative activity (e.g., anti-tumor potentiating activity). In one embodiment, fractions A, B and C are combined in approximately equal proportions by mass. In another embodiment, fractions A, B and C are combined in unequal proportions by mass. Any one of the fractions can be proportionally greater than or less than any of the other fractions, for example, the ratio of A:B:C can be 1:1:2, 1:2:1, 2:1:1; 1:1:3, 1:3:1, 3:1:1; 1:1:4, 1:4:1, 4:1:1; 1:1:5, 1:5:1, 5:1:1; 1:2:2; 2:2:1; 1:3:3; 3:3:1; or a fraction, e.g., 1.5:1:1; or a relatively larger proportion of one fraction in comparison to other(s), e.g., 20:1:1, 20:20:1, 100:1:1, 100:100:1, etc.

Methods and compositions of the invention additionally include fractions denoted A, B and C alone, and in combinations of two or more fractions. Single fractions and combinations can be combined in any proportion so long as fractionating the single fraction or combination with a non polar solvent produces a composition having one or more activities described herein, for example, immune-stimulating, myelosuppression inhibiting or anti-cell proliferative (e.g., anti-tumor) potentiating activity. The fractions may be fractionated prior to or after combining in the desired proportion. In one embodiment, fractions A and B are combined. In another embodiment, fractions A and C are combined. In an additional embodiment, fractions B and C are combined. In yet additional embodiments, fractions A and B, A and C, and B and C are combined in equal and unequal proportions by mass. Any one of the fractions can be proportionally greater than or less than any of the other fractions, such as, the ratio can be 1:2, 1:3,1:4, 1:5, 1:6, etc., or a fraction, e.g., 1.5:1, 2.5:1, etc., or a large proportion of one fraction in comparison to the other fraction, e.g., 20:1, 50:1, 100:1, 500:1, etc.

Fractions A, B and C are fractionated in order to obtain a composition having immune stimulating or anti-cell proliferative activity. Typically, the combination or single fraction has one or more impurities, toxins, or inhibitory compounds removed by the fractionation. For example, alkaloids are present in one or more of fractions A, B and C and are toxic to animals. Free withanolides may also be present in one or more of fractions A, B and C. Accordingly, in one embodiment, the fractionation removes one or more alkaloids from the combined or a single fraction. In another embodiment, the fractionation removes one or more withanolides from the combined or a single fraction.

Suitable solvents for this particular fractionation step are typically non polar and include, for example, methylene chloride (polarity index 3.1), diethyl ether (polarity index 2.8) or chloroform (polarity index 4.1). Other non polar solvents and solvents within the same chemical class of methylene chloride, diethyl ether or chloroform are known in the art and are applicable in the fractionation (see, e.g., Merck Index).

Thus, the invention further provides compositions obtained from Withania Somnifera characterized as having immune stimulating activity or anti-tumor activity (including anti-tumor potentiating activity). In one embodiment, a composition is soluble in water; substantially or completely free of alkaloids; and having at least one glycowithanolide. In other embodiments, a composition is further characterized as having a glycowithanolide content ranging from about 0.5 to 1.6%, 0.5 to 2.5% or 0.5 to 5% by weight. In yet another embodiment, a composition is further characterized as substantially or completely free of withanolides.

A used herein, the term “substantially free” means that the component so referred to is present in small amounts, typically 5% or less by weight (e.g., 4%, 3%, 2%, 1% or less). The term “free” means that the component so referred is present in undetectable amounts (within the limits of detection for the detection method), and therefore, may still be present in minute quantity, but is below the level of detection. For example, glycowithanolides can be detected in amounts as low as 0.05% using the assay described in Example I. Absence or presence of alkaloids can be determined, for example, by negative tests using dragendorff's reagent and Myers reagent, each which have a detection limit of a few micrograms.

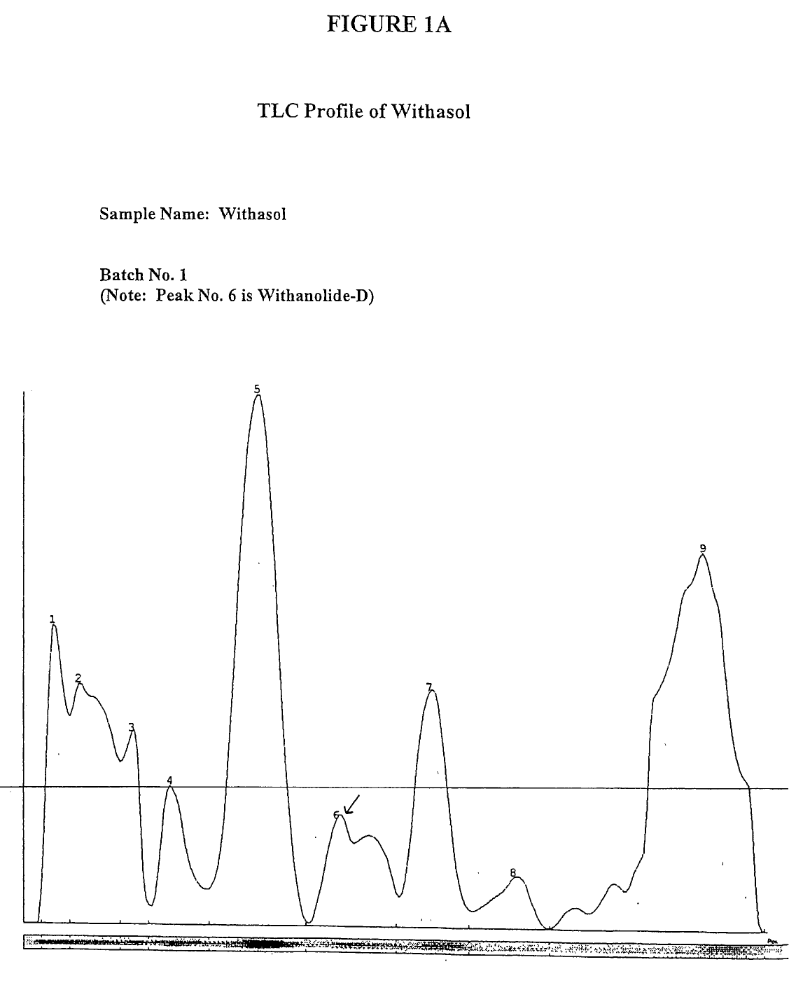

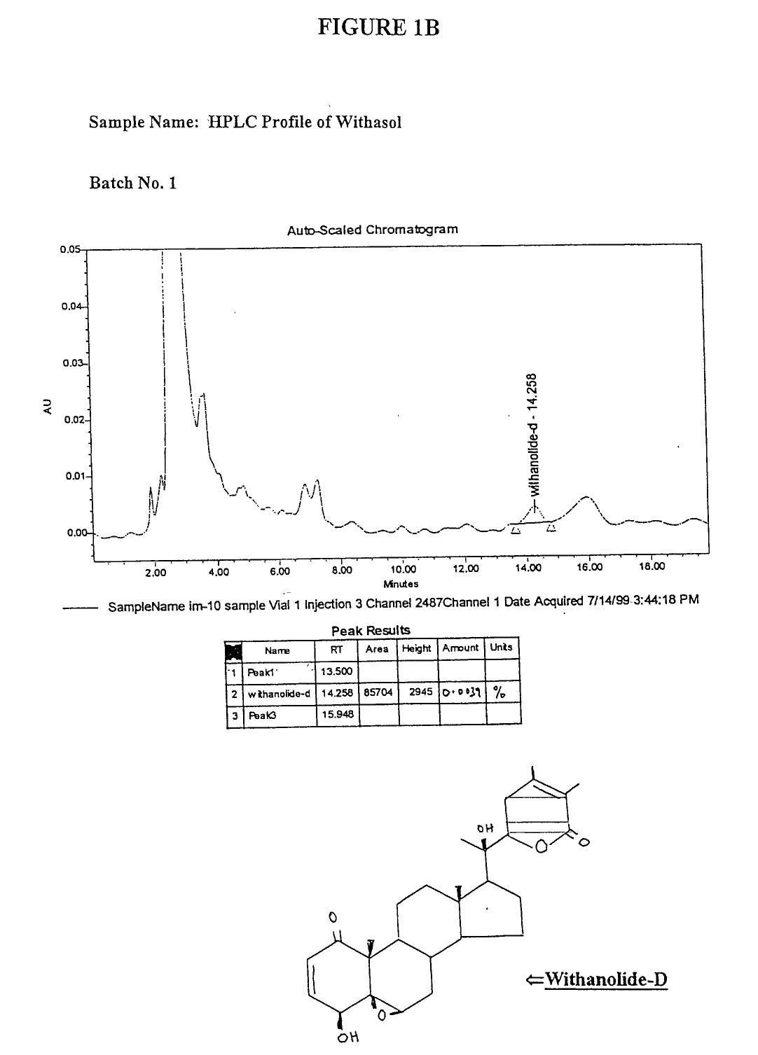

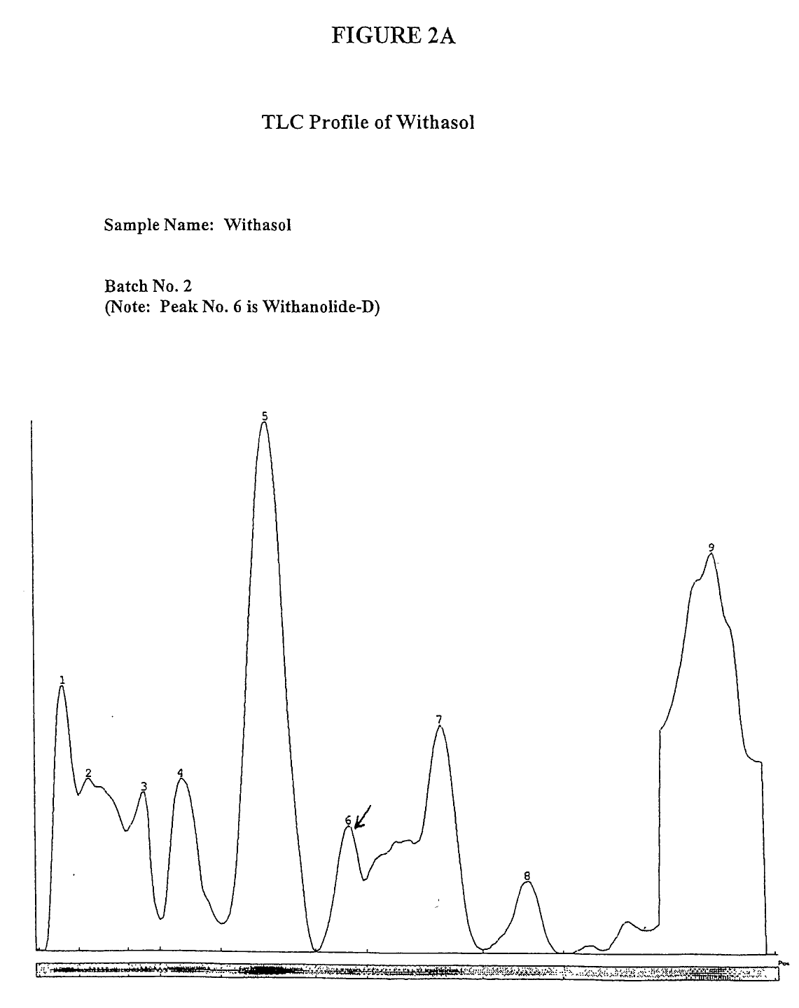

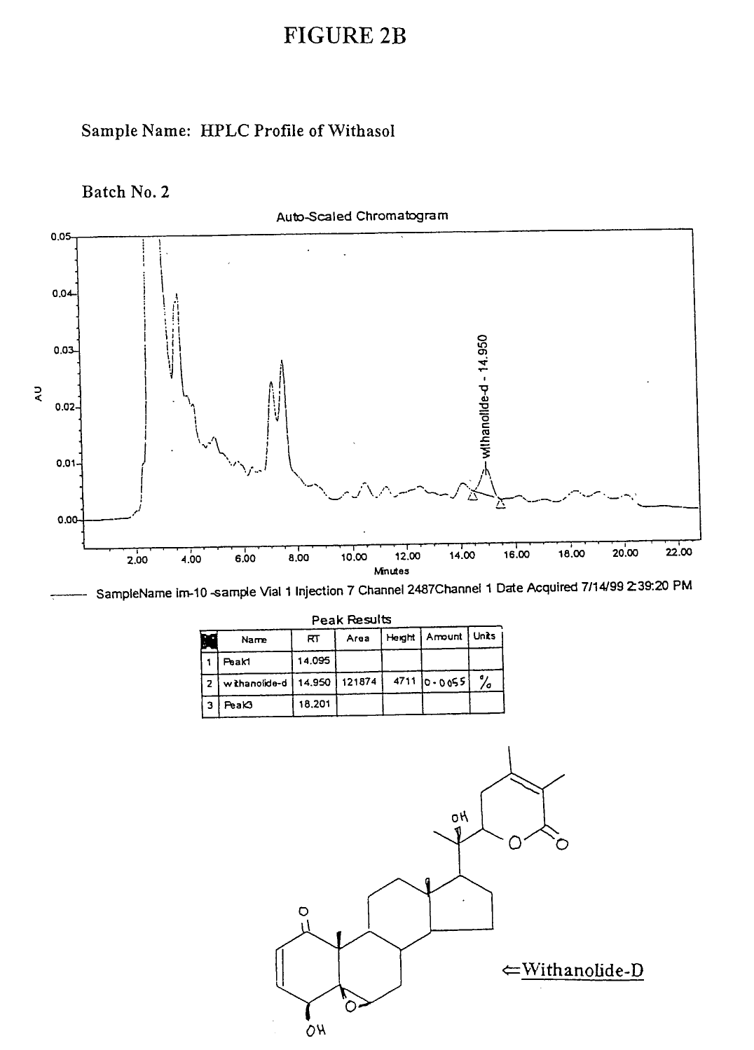

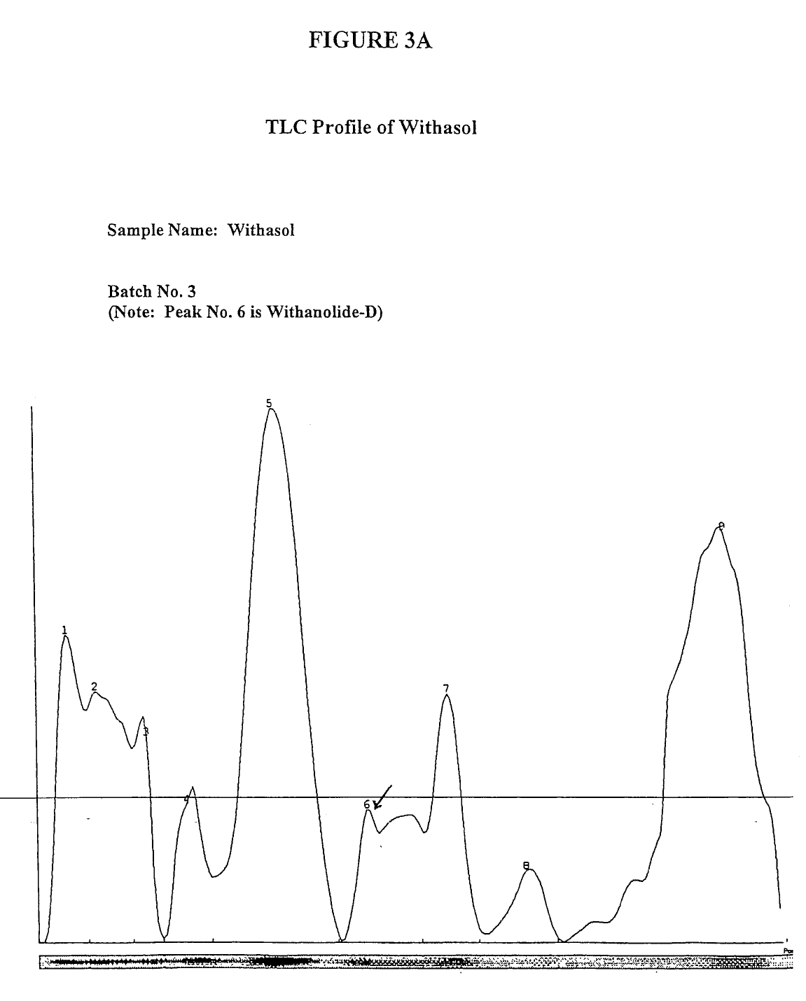

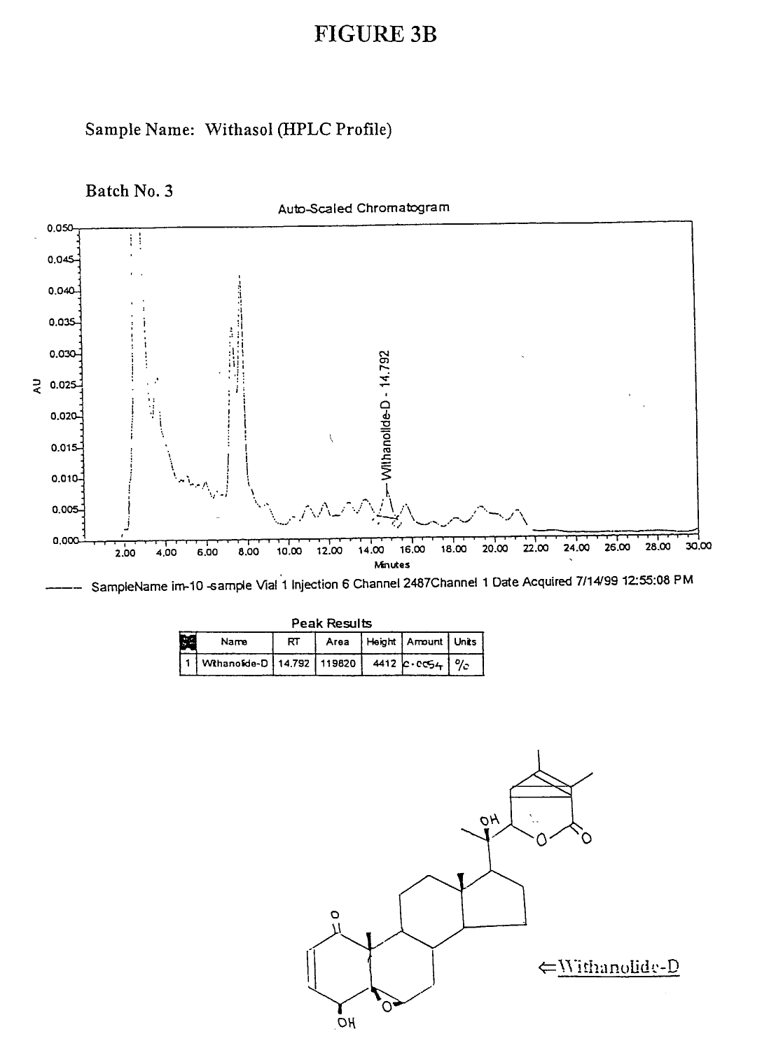

In another embodiment, a composition of the invention is characterized as having a TLC profile substantially the same as a profile set forth in FIGS. 1A, 2A or 3A, the profile obtained with a hexane:methylene chloride:methanol mobile phase in about a 20:30:2 ratio. In still another embodiment, a composition of the invention is characterized as having an HPLC profile substantially the same as a profile set forth in FIGS. 1B, 2B or 3B, said profile obtained using a reverse-phase C-18 column at a flow rate of about 1.0 ml/min with a mobile phase of methanol:water in a ratio of about 60:40.

As used herein, the term “substantially the same,” when used in reference to a composition having a TLC or HPLC profile exemplified in FIGS. 1 to 3, means that each peak present in a particular preparation has an Rf value within the standard deviation shown for a reference composition, such as that shown in Table 1. For example, in the TLC there are 9 major peaks total (peaks 1 to 5 and 7 to 9; peak 6 is withanolide D). Peak 6 has an Rf value of about 0.34 with a standard deviation of 0.0322. Thus, a peak 6 with an Rf from about 0.3722 to about 0.3078 is considered substantially the same. A composition substantially the same can also be measured by the area of a peak. Thus, in the example of peak 5 the mean percent area is 29.9 and a composition substantially the same will be within the standard deviation or relative standard deviations shown in Table 1. Similarly, for HPLC, retention times, standard deviation and relative standard deviation can be used as guides to indicate whether a composition is substantially the same. HPLC retention times, mean retention time, standard deviation and relative standard deviations for batch numbers 2 and 3 are shown in Table 2. Of course, there can be less variation in Rf value, peak area, retention time, etc., for a particular preparation of an invention composition.

To demonstrate batch-to-batch consistency of Withasol preparations nine major peaks of each batch of the same Rf values were taken for comparison with respect to their % areas. The mean % area, standard deviation and % relative standard deviation (RSD) are given in Table 1.

TABLE 1
TLC profile of Withasol Batches

| | | | | Batch | Batch | Batch | Mean | | |
|---|---|---|---|---|---|---|---|---|---|
| Peak | Mean | Std. | | no. 1 | no. 2 | no. 3 | % | Std. | |
| No. | Rf | Dev. | RSD | % Area | % Area | % Area | Area | Dev. | RSD |
| 1 | 0.1 | 0.002 | 2.0 | 14.52 | 9.83 | 7.25 | 10.53 | 3.7 | 35 |
| 2 | 0.14 | 0.118 | 8.4 | 6.23 | 5.79 | 7.26 | 6.43 | 0.75 | 11.7 |
| 3 | 0.17 | 0.014 | 8.1 | 5.12 | 5.64 | 5.19 | 5.32 | 0.28 | 5.31 |
| 4 | 0.22 | 0.0065 | 3.0 | 10.52 | 9.86 | 10.24 | 10.21 | 0.33 | 3.25 |
| 5 | 0.31 | 0.0055 | 1.8 | 29 | 33.7 | 27.1 | 29.9 | 3.36 | 11.2 |
| 6 | 0.34 | 0.0322 | 9.3 | 22.03 | 22.61 | 21.85 | 22.16 | 0.397 | 1.79 |
| 7 | 0.48 | 0.0033 | 0.7 | 8.14 | 15.98 | 16.39 | 13.5 | 4.65 | 34.4 |
| 8 | 0.54 | 0.026 | 4.8 | 5.29 | 5.55 | 5.29 | 5.38 | 0.15 | 2.8 |
| 9 | 0.97 | 0.006 | 0.6 | 28.65 | 29.05 | 27.36 | 28.35 | 0.883 | 3.11 |

TABLE 2
Withasol HPLC Chromatograms Retention time data

| RT of | RT of | RT of | | | |
|---|---|---|---|---|---|
| Batch No. 1 | Batch No. 2 | Batch No. 3 | Mean RT | Std. Dev. | RSD |
| 3.905 | 3.905 | 3.887 | 3.90 | 0.10 | 0.27 |
| 4.517 | 4.726 | 4.735 | 4.66 | 0.123 | 2.65 |
| 5.698 | 5.696 | 5.676 | 5.69 | 0.012 | 0.21 |
| 6.677 | 6.666 | 6.69 | 6.68 | 0.012 | 0.18 |
| 8.385 | 8.378 | 8.345 | 8.37 | 0.021 | 0.26 |
| 9.668 | 9.669 | 9.64 | 9.66 | 0.016 | 0.17 |
| 13.089 | 13.1 | 13.079 | 13.09 | 0.011 | 0.08 |
| 24.619 | 24.864 | 24.591 | 24.69 | 0.15 | 0.61 |

In another embodiment, a composition includes a glycowithanolide comprising sitoindoside IX. In yet another embodiment, a composition includes a glycowithanolide comprising sitoindoside X. In still another embodiment, a composition includes a glycowithanolide comprising a mixture of sitoindoside IX and sitoindoside X. In yet additional embodiments, a composition includes one or more glycowithanolides distinct from sitoindoside IX and sitoindoside X; two or more glycowithanolides distinct from sitoindoside IX and sitoindoside X; and three or more glycowithanolides distinct from sitoindoside IX and sitoindoside X. Compositions further include sitoindoside molecules having R group substitutions that extend from the ring structure of the molecule.

A composition of the invention can be further characterized as having one or more of the following by mass: about 35-75% protein content; about 0.5 to about 5% glycowithanolide(s); about 3 to about 10% ash; and about 30 to about 60% carbohydrate.

Compositions of the invention can also be characterized by their biological activity. For example, a composition can be characterized by the presence or amount of immune stimulating (e.g., increases in white blood cells or antibody response), myelosuppression inhibiting (e.g., reverses, at least in part, decreases of white cell numbers) or anti-cell proliferative activity (e.g., anti-tumor potentiating activity). In one embodiment, administering about 100 mg/kg subject mass of a composition to a subject increases by about 10% or more the number of white blood cells in the subject. In another embodiment, administering about 100 mg/kg subject mass of a composition to a subject increases by about 20% or more the number of white blood cells in the subject.

Increases in immune response characterized by increases in the number of cells associated with immune-responsiveness, such as neutrophils, following treatment of a subject with a composition of the invention, are typically between about 20% to 50% above the number prior to treatment, more typically 35% to 50%. It is noted however that the response may be more or less in individual subjects due to variation between individuals in their particular immune responsiveness at the time of treatment with an invention composition (e.g., higher than normal, normal, or less than normal), pharmacological activity of the composition, circulating half-life of the composition, or elimination efficiency of the composition. For example, increases in number of blood cells (e.g., white cells) in an individual subject that is immunosuppressed are expected to be greater than in a comparable subject that is not immunosuppressed. Thus, increases in the number of blood cells can be more, for example, from about 30% to 40%, 40% to 50%; or less, for example, from about 15% to 20%, 10% to 15%, or less, for example, 5% to 10%, above the number prior to treatment.

Increases in cell numbers in individual subjects may be broad in spectrum, and may include myeloid and lymphoid cell types, or may be observed in particular subsets of immune cells. For example, the increases in individual subjects may be limited to particular cell lineages (e.g., myeloid or lymphoid), or types/subtypes of cells such as lymphocytes, B cells, T cells or natural killer cells; for monocytes, macrophages, antigen-presenting cells (e.g., dendritic cells), Kupffer cells, Langerhans cells, microglial cells or mesangial cells; for leukocytes/granulocytes, neutrophils, basophils, eosinophils or mast cells, etc.

Changes in cell number for the various cell types can be determined as described, for example, in Example III. In brief, blood cells are counted with standard calibrating systems in such as a Coulter counter or Sysmex K-1000 cell counter. These and other instruments are able to distinguish between all white cell types and their precursor cells. In case of abnormal counts or to confirm accuracy of results, the counts can be cross checked manually by doing a peripheral smear under the microscope.

Normal Ranges for White Blood Cells are as follows:

| | |
|---|---|
| Total White Count | 2500-7500 cells/mm³ |
| Neutrophuls | 1830-7250 cells/mm³ |
| Lymphocytes | 1500-4000 cells/mm³ |
| Monoytes | 200-950 cells/mm³ |
| Eosinophils | 0-700 cells/mm³ |
| Basophils | 0-150 cells/mm³ |

Increases in immune response characterized by increases in antibody-mediated response (e.g., HA or HL titres) will be between about 20% to 30% above the response prior to treatment with a composition of the invention. As discussed, depending on the individual subject, increases in antibody-mediated response can be more, for example, from about 30% to 50%; or less, for example, from about 15% to 20%, 10% to 15%, 5% to 10%, above the antibody-mediated response prior to treatment with a composition of the invention. Changes in antibody-mediated response can be determined by HA or HL titre assays, an increase in either one of which establishes activity of an invention composition (see, e.g., Example III).

As used herein, the term “subject” refers to an animal. Typically, the animal is a mammal, however, any animal (e.g., bird, reptile, etc.) capable of exhibiting humoral or cell-mediated immunity is included within the term. Particular examples of mammals are primates (humans, apes, chimps, macaques), domestic animals (e.g., dogs, cats), livestock (e.g., horses, cattle, pigs, sheep, goats, etc.) and rodents (e.g. rats, mice, rabbits).

A subject as defined herein can be normal with respect to immune-responsiveness (i.e., not immunosuppressed or myelosuppressed), be immunosuppressed or myelosuppressed, be at risk of immunosuppression or myelosuppression, have a hyperactive immune response that either causes autoimmunity or anergy, or be at risk of a hyperactive immune response. These various states of immune response can be naturally occurring or result from therapies that directly or indirectly cause them. In addition, the various states of a subject's immune response can result from environmental exposure. For example, myelosuppression or immunosuppression may result directly or indirectly from anti-cell proliferative or anti-cancer (e.g., radiation, radioisotopes, chemotherapy, etc.) therapy, or steroidal and other therapies known in the art, or radiation or a toxin present in the environment, industrial exposure, etc. The subject can be myelosuppressed, or at risk of myelosuppression.

A subject as defined herein can also be at risk of a hyperproliferative disorder, have a hyperproliferative disorder, or is in remission from or previously had a hyperproliferative disorder. A subject can be at risk of undergoing an anti-cell proliferative therapy, undergoing an anti-cell proliferative therapy, or previously have undergone an anti-cell proliferative therapy. A hyperproliferative subject includes neoplasia, or benign hyperproliferation.

A composition of the invention obtained from fractions A, B and C alone, or in any combination, after fractionation with a nonpolar solvent, can be further purified into subfractions with other components therein and inert substances removed. As discussed, further purification can remove one or more alkaloids or one or more withanolides. Such further purified subfractions can contain one or more glycowithanolide (e.g., sitoindoside IX, sitoindoside X, etc.) having immune-stimulating or anti-tumor potentiating activity.

Subfractions can be obtained by biochemical purification and fractionation of an invention composition as described herein, such as size-based molecular fractionation, for example, size exclusion by gel filtration or by membrane filtration using a membrane having pore sizes through which only molecules having less than a certain size may pass, gel electrophoresis and sucrose gradient centrifugation. Fractionation techniques also include techniques that separate molecules on the basis of physical or chemical characteristics (e.g., hydrophobicity, hydrophilicity, polarity, affinity, etc.), which include, for example, chromatography, thin layer chromatography (TLC), high-pressure liquid chromatography (HPLC), cation exchange (strong or weak), FPLC, reverse phase, hydrophobic interaction, and affinity (metal-chelate, antibody, ligand, receptor, etc.) chromatography. Fractionating the composition in order to obtain subfractions can be performed with any of a variety of established techniques known in the art; appropriate media for such fractionations are commercially available (see, e.g., Remington's Pharmaceutical Sciences (1990) 18th ed., Mack Publishing Co., Easton, Pa.; Pharmacia Biotech BioDirectory '96).

Although a subfraction will generally be distinct from the composition from which it is obtained (parent composition), a subfraction will have one or more components or subcomponents in common with the parent composition. For example, where a subfraction is prepared from WITHASOL, the subfraction can contain one or more components comprising one or more TLC spots or HPLC peaks of WITHASOL, as exemplified in FIGS. 1 to 3. As there may be multiple components within a peak, the subfraction may contain one or more such subcomponents. Subfractions, or combinations of subfractions, will therefore likely have immune-stimulating or anti-cell proliferative activity. Further purifications of invention compositions, including WITHASOL, having immune-stimulating or anti-cell proliferative activity are therefore explicitly included. Subfractions, including a particular spot, or combination of spots, isolated from TLC or HPLC peak or combination of peaks (e.g., a subfraction), for example, can be examined for having immune-stimulating or anti-cell proliferative activity using the animal studies described herein. In vivo tests, including human clinical trials for establishing immune stimulating anti-cell proliferative activity are described, for example, in Metcalf D. Blood 67(2):257-267 (1986); Row et al,. Blood 86:457-462 (1995); Morstyn et al,. Lancet. 1:667-672 (1988); Bronchud et al,. Br J Cancer 56:809-813 (1987); Neidhart et al,. J Clin Oncol. 7:1685-1691 (1988); and Dale et al,. Blood 81:2496-2502 (1993).

Thus, in accordance with the invention, there are provided compositions, including subfractions and subfraction combinations, having immune stimulating or anti-tumor activity, purified in addition to the purified fractions exemplified herein. In one embodiment, a composition includes any one of TLC peaks 1 to 9 set forth in FIGS. 1A, 2A or 3A alone, or in any combination with each other. In another embodiment, a composition includes one or more of the molecules in TLC spots 1 to 9 set forth in FIGS. 1A, 2A or 3A alone, or in any combination with each other. In yet another embodiment, a composition includes any one of HPLC peaks set forth in FIGS. 1B, 2B or 3B alone, or in any combination with each other. In yet another embodiment, a composition includes any one of the molecules in HPLC peaks set forth in FIGS. 1B, 2B or 3B alone, or in any combination with each other.

As discussed, in order to identify active subfractions, immune-stimulating or anti-cell proliferative activity can be tested as illustrated in Example IV. For example, a panel of TLC spots, such as peaks 1 to 3, or 4 and 5, or 6 to 9, can be isolated from the TLC plate and, administered alone, or in any combination to the cyclophosphamide treated mouse implanted with ascitic sarcoma (S-180) in order to identify subfractions having immune-stimulating, anti-myelosuppressive or anti-cell proliferative activity. For example, a subfraction that increases the number of white blood cells or antibody response (e.g., HA or HL titre) would identify the subfraction as having immune-stimulating activity. A subfraction that increases macrophages, antigen-presenting cells, dendritic cells, Kupffer cells, Langerhans cells, microglial cells or mesangial cells would identify the subfraction as having anti-myelosuppressive activity. A subfraction that decreases tumor size (e.g., decrease in animal weight) or decreases tumor growth, or reduces the rate at which the tumor grows would identify the subfraction as having anti-cell proliferative activity. Similarly, an HPLC peak, or combination of peaks, can be isolated and administered to a cyclophosphamide treated mice implanted with ascitic sarcoma (S-180) in order to identify HPLC subfraction(s) having immune-stimulating, anti-myelosuppressive or anti-cell proliferative activity.

Thus, the invention further provides methods for identifying subfractions of invention compositions having the requisite activity, including subfractions of the exemplified WITHASOL. In one embodiment, a method of the invention includes contacting an animal with a subfraction (e.g., TLC or HPLC subfraction as set forth in FIGS. 1-3 or defined in Tables 1 and 2) and determining whether the subfraction has immune-stimulating activity in the animal. In another embodiment, a method of the invention includes contacting an animal with a subfraction and determining whether the subfraction has anti-myclosuppressive activity. In yet another embodiment, a method of the invention includes contacting an animal with a subfraction and determining whether the subfraction has anti-cell proliferative activity (e.g., anti-tumor potentiating activity). In various additional embodiments, the subfractions are obtained from TLC and HPLC fractionated WITHASOL. In further embodiments, the subfractions are obtained from fractions A, B and C alone, or A, B and C in any combination with each other, before or after fractionation with a non polar solvent.

Invention compositions can have immune stimulating or anti-myelosuppressing activity. For example, WITHASOL, when administered to cyclophosphamide treated tumor bearing animals stimulated immune response and reversed the myelosuppression caused by cyclophosphamide treatment (see, e.g., Example IV). Invention compositions are therefore useful in reversing, at least in part, immunosuppression caused by the use of immunosuppressing drugs. In addition, invention compositions can have anti-cell proliferative activity, particularly when use in combination with an anti-cell proliferative treatment regimen. For example, WITHASOL, when administered in combination with cyclophosphamide to treat tumor bearing animals, produced a greater reduction in tumor size than the reduction of the tumor in the animals treated with cyclophosphamide alone (see, e.g., Example IV).

Thus, in accordance with the invention, there are further provided pharmaceutical formulations including an invention composition. In one embodiment, a pharmaceutical formulation includes WITHASOL. In another embodiment, a pharmaceutical formulation includes an invention composition in combination with one or more other drugs. In one aspect, the drug comprises an immune-stimulating drug. In another aspect, the drug comprises an anti-myelosuppressive drug. In yet another aspect, the drug comprises an antibody response-stimulating drug. In still another aspect, the drug comprises an immunosuppressive drug. In a further aspect, the drug comprises an anti-cell proliferative drug. Such formulations can be administered to a subject in vivo in order to practice the treatment methods of the invention, for example.

Compositions of the invention, including additionally purified forms and subfractions thereof, can be formulated into pharmaceutical formulations appropriate for internal or external administration. The pharmaceutical formulations will be in a “pharmaceutically acceptable” or “physiologically acceptable” form. As used herein, the terms “pharmaceutically acceptable” and “physiologically acceptable” refer to carriers, diluents, excipients, and other preparations that can be administered to a subject, without destroying activity or adsorption of an invention composition.

Invention pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like. In one embodiment, a pharmaceutical formulation is suitable for oral consumption. In one aspect, a pharmaceutical formulation includes a pill (tablet or capsule). In another aspect, a pharmaceutical formulation includes a syrup or elixir with another suitable liquid, e.g., glycerol, sorbitol or sucrose.

A pharmaceutical formulation can be formulated to be compatible with its intended route of administration. Thus, in additional embodiments, a pharmaceutical formulation includes carriers, diluents, or excipients suitable for administration by routes including intraperitoneal, intramuscular, intradermal, subcutaneous, oral, intranasal (e.g., inhalation), intravenous, intracavity, intracranial, transdermal (topical), parenteral, e.g. transmucosal, and rectal administration and intra-tumoral administration.

Solutions or suspensions used for parenteral, intradermal, or subcutaneous application can include the following: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. Acids or bases, such as hydrochloric acid or sodium hydroxide can be used to adjust pH. The parenteral preparation can be enclosed in ampules, disposable syringes or multiple dose vials made of glass or plastic.

Pharmaceutical formulations suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and be fluid to the extent that syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, sodium chloride will be included in the composition. Prolonged absorption of injectable formulations can be achieved by including an agent that delays absorption, for example, aluminum monostearate or gelatin.

Sterile injectable solutions can be prepared by incorporating the composition in the required amount in an appropriate solvent with one or a combination of ingredients enumerated above, as required, followed by filtered sterilization. Generally, dispersions are prepared by incorporating the active composition into a sterile vehicle that contains a basic dispersion medium and other ingredients from those enumerated above or known in the art.

Oral formulations generally include an inert diluent or an edible carrier. For the purpose of oral therapeutic administration, a composition can be incorporated with excipients and used in the form of tablets, troches, or capsules, e.g., gelatin capsules.

Pharmaceutically compatible binding agents, and/or adjuvant materials can be included in the formulation. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the formulation can be delivered via a pump or an aerosol spray from a dispenser or pressured container that contains a suitable propellant, e.g., a gas such as carbon dioxide, or a nebulizer.

Systemic administration can be achieved, inter alia, by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, compositions can be formulated into ointments, salves, gels, or creams as generally known in the art.

Targeted administration can be achieved by injection or an implantable device located in or near the target cells or tissue. For example, the formulation can be administered by infusion into the target over time or via a bolus.

Formulations can also include carriers to protect the composition against rapid degradation or elimination from the body, such as a controlled release formulation, including implants and microencapsulated delivery systems. Tablets may be formulated or coated to delay disintegration or absorption in the gastrointestinal tract for sustained action over a longer period of time. For example, a time delay material such as glyceryl monostearate or glyceryl stearate alone, or in combination with a wax, may be employed.

Additional formulations include incorporating the composition into biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. Methods for preparation of such formulations will be apparent to those skilled in the art. The materials can also be obtained commercially from Alza Corporation and Nova Pharmaceuticals, Inc.

The rate of release of a composition can be controlled by altering the concentration or composition of such macromolecules. For example, the composition can be entrapped in microcapsules prepared by coacervation techniques or by interfacial polymerization, for example, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems including oil-in-water emulsions, micelles, mixed micelles, and liposomes. These can be prepared according to methods known to those skilled in the art (for example, as described in U.S. Pat. No. 4,522,811).

Formulations further include unit dosage forms for convenient administration and uniformity of dosage. “Unit dosage forms” as used herein refer to physically discrete units suited as unitary dosages for the subject to be treated; each unit containing a predetermined quantity of composition determined to produce a desired therapeutic effect in association with the required pharmaceutical carrier.

Additional pharmaceutical formulations appropriate for administration are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

Kits including the pharmaceutical formulations of the invention also are provided. Such kits are useful for practicing the treatment methods of the invention. In one embodiment, a kit of the invention contains WITHASOL, and a label or packaging insert for stimulating immune response, inhibiting myelosuppression, or treating a cell proliferative disorder or condition, in suitable packaging material. As used herein, the term “packaging material” refers to a physical structure housing the components of the kit, such as invention fractions, subfractions, extracts, etc. The packaging material can maintain the components sterilely, and can be made of material commonly used for such purposes (e.g., paper, corrugated fiber, glass, plastic, foil, etc.). The kit can contain unit dosage forms of the pharmaceutical formulations (e.g., in ampules) and appropriate instructions. The label or packaging insert can indicate that the kit is to be used in a method of the invention.

Pharmaceutical formulations including an invention composition can include other drugs, therapeutic agents and herbal medicines. Such additional drugs, therapeutic agents and herbal medicines can provide an additive or synergistic effect when used in combination with an invention composition having immune-stimulating, anti-myelosuppressing or anti-cell proliferative activity thereby enhancing one or more of immune-stimulating, anti-myelosuppressing or anti-cell proliferative activities. Drugs, therapeutic agents and herbal medicines also included are those that produce undesirable side effects that can be reversed, at least in part, by an invention composition. Thus, a drug that directly or indirectly causes immunosuppression or myelosuppression, for example, as a side effect, can be combined with an invention composition in order to inhibit undesirable immunosuppression or myelosuppression. For example, an invention composition can be included with an anti-cell proliferative drug such as cyclophosphamide in order to counteract cyclophosphamide induced immunosuppression or myelosuppression.

As used herein, the terms “drug,” “agent,” or “medicine” are used interchangeably and include any molecule, natural or synthetic, having a biological activity including, for example, small organic molecules, herbal mixtures (e.g., purified and crude extracts), radioisotopes, polypeptides (growth factors, signaling molecules, receptors, antibodies, receptor ligands, etc.), peptidomimetics, nucleic acids (coding for polypeptide or antisense) or fragments thereof. Organic drugs or agents often comprise cyclical carbon or heterocyclic structures, and/or aromatic or polyaromatic structures substituted with one or more functional groups. Drugs or agents are also found among biomolecules, including, but not limited to, saccharides, fatty acids, hormones, vitamins, steroids, purines, pyrimidines, derivatives, structural analogs, or combinations thereof. Known pharmacological drugs and agents are also included and can be found, for example, in Physicians' Desk Reference (1997) 51^{st} ed., Medical Economics Company, Inc., Montvale, N.J.; The Pharmacological Basis of Therapeutics, J. G. Hardman and L. E. Limbird, eds. (1996) Ninth ed., McGraw-Hill, N.Y.

In additional embodiments, invention compositions, including pharmaceutical formulations, are packaged within a container, pack or dispenser, optionally sterile, together with instructions for use. The instructions are appropriate for practicing a method of the invention. In various aspects, instructions include instructions for treating immune suppression, myelosuppression or abnormal or undesirable cell proliferation. Instructions may be printed, such as on paper, or contained in a computer readable medium.

Thus, the invention further provides compositions including an invention composition in combination with one or more additional therapeutic agents, e.g., other immune stimulating or suppressing, other anti-myelosuppressing or stimulating and other anti-cell proliferative drugs or agents. In one embodiment, a composition includes an immunomodulating drug. In one aspect, a composition includes a drug that has immunostimulating activity. In another aspect, a composition includes a drug that increases white blood cell numbers in a subject. In another embodiment, a composition includes a drug that increases antibody response in a subject. In yet another embodiment, a composition includes a drug that has immunosuppressing activity in a subject. In still yet another embodiment, a composition includes a drug that produces myelosuppression or decreases antibody response in a subject. In additional embodiments, a composition includes a drug that activates or primes immune response cells, stimulates immune cell proliferation or modulates immune cell chemotaxis. In various aspects, a drug is a cytokine or a chemokine, for example, an interleukin (e.g., IL-1), interferon, GM-CSF, TNF-α, TNF-β, etc.

Specific examples of immunostimulating drugs include muramyl dipeptide, isoprinosine, leflunomide, aldesleukin, synthetic polyribonucleotides and interferon inducers such as tilerone. Levamisole, an anti-helminthic agent, enhances immunity to infectious agents in animals. Specific examples of immunosuppressing drugs include cyclosporin, tacrolimus, mycophenolate mofetil, mycophenolic acid and corticosteroids (e.g., prednisone, prednisolone, dexamethasone, triamcinolone).

In another embodiment, a composition includes an invention composition in combination with a drug that has anti-cell proliferative activity. In various aspects, a composition includes a drug that inhibits cell cycle progression, cell proliferation or nucleic acid or protein synthesis. In yet another embodiment, a composition includes an anti-neoplastic drug or a steroid glycoside (i.e., a steroid having a sugar molecule, such as glucose, rhamnose or galactose, attached). In one aspect, an anti-neoplastic drug comprises a chemotherapeutic agent.

As used herein, the term “anti-cell proliferative” and grammatical variations thereof means reducing the number of cells, preventing increases in numbers of cells or inhibiting increases in numbers of cells. This activity may occur due to decreasing the amount of cell proliferation or decreasing the survival time of the cells. For example, if cells are not actively proliferating but survive for a longer period of time (e.g., instead of dying in days, they live for months or longer), this may result in an overall increase in the number of cells without proliferation occurring. Accordingly, the term refers to a reduction in cell numbers or a lessening of increasing numbers of cells. Thus, an invention composition that has “anti-cell proliferative” activity decreases cell numbers, prevents increases in cell numbers or inhibits the rate of increase in cell numbers (e.g., slows or stops the increase in cell numbers that occurs due to increased cell proliferation or cell survival) when the physiological condition is such that the cell numbers are increasing, as in a growing neoplasia, for example.

As used herein, the terms “anti-neoplastic activity” and “anti-tumor activity” mean anti-cell proliferative activity where the cells are neoplastic or tumerous. The terms “anti-cell proliferative potentiating activity” and “anti-tumor potentiating activity” mean enhancing anti-cell proliferative activity when used in combination with any anti-cell proliferative therapy, and enhancing anti-tumor potentiating activity when used in combination with anti-cell proliferative activity where the target cells are neoplastic or tumorous, respectively.

Particular anti-proliferative agent or drug activities include inhibiting progression through the cell cycle or cell proliferation, stimulating apoptosis or cell death, inhibiting nucleic acid or protein synthesis or metabolism, inhibiting cell division or inhibiting production or utilization of a necessary cell survival factor, growth factor or signaling pathway (extracellular or intracellular). Particular examples of chemical agents having anti-cell proliferative (including anti-cell proliferative potentiating) and anti-tumor (including anti-tumor potentiating) activities useful in the compositions and methods of the invention include alkylating agents, anti-metabolites, plant alkaloids, plant extracts, antibiotics, nitrosoureas, hormones, nucleoside analogues and nucleotide analogues. Particular examples of drugs include cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-AZC, taxol, vinblastine, vincristine, doxorubicin, bleomycin, actinomycin D, mithramycin, mitomycin C, carmustine, lomustine, semustine, streptozotocin, hydroxyurea, cisplatin, mitotane, procarbazine, dacarbazine and dibromomannitol.

As discussed, invention compositions can have immune stimulating or anti-myelosuppressing activity. Invention compositions, including further purified forms and subfractions thereof, are therefore useful for treating subjects having or at risk of having various disorders or physiological conditions characterized by abnormal or undesirable immune suppression or myelosuppression. Invention compositions also have applicability in stimulating immune response in subjects even if their response is within the normal range. For example, a composition that stimulates immune response can be administered to a subject in order to increase efficacy of a vaccine (e.g., increased antibody titre).

Thus, in accordance with the invention, there are also provided methods for treating a subject with a composition of the invention. In one embodiment, a method includes administering a composition to the subject to increase immune response. In one aspect, the subject has or is at risk of having immunosuppression (e.g., less than normal numbers of white blood cell types, antibody titre). In another aspect, the composition increases the number of white blood cells in the subject. In yet another aspect, the subject is currently undergoing or will be undergoing an immunosuppressive therapy. In another embodiment, a method includes administering a composition to the subject to inhibit myelosuppression. In one aspect, the subject has or is at risk of having myelosuppression.

Immune disorders or conditions amenable to treatment include immunodeficiency caused by a genetic abnormality, an infectious disease (e.g., an immunosuppressing disease such as HIV) or physiological disorder, or an exogenous environmental or therapeutic exposure to a chemical or other immune system insult. Generally, immunodeficiency results in a decreased ability of a subject to mount a primary immune response due to inhibition of antigen processing, immune cell production/proliferation/activation or chemokine/cytokine synthesis, signaling or response. A subject suffering from immunodeficiency can therefore be treated. Specific examples of appropriate subjects include those undergoing a therapy that directly or indirectly causes immunosuppression or myelosuppression, such as cancer treatment, radiation treatment, chemotherapy or radioisotope treatment (where the chemotherapeutic agent or radioisotope is administered to the subject), or bone marrow transplantation.

Immune disorders or conditions amenable to treatment also include subjects in which hypersensitivity or an undesirably active immune response is caused by a deficiency in immune cells that down regulate/attenuate the immune response. A decreased number of cells that down regulate or attenuate an immune response can lead to immune hypersensitivity. Thus, a subject having hypersensitivity of an immune response can be treated with a composition of the invention in order to increase the number of cells, or activate the cells that attenuate the immune response, in order to reduce or eliminate immune hypersensitivity. Such diseases include, for example, asthma, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, Crohn's disease, ulcerative colitis, aphthous ulcer, iritis, conjunctivitis, keratoconjunctivitis, asthma, allergic asthma, cutaneous lupus erythematosus, scleroderma, vaginitis, proctitis, autoimmune uveitis, allergic encephalomyelitis, acute necrotizing hemorrhagic encephalopathy, idiopathic bilateral progressive sensorineural hearing loss, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), and allergy such as, atopic allergy. In addition, a subject undergoing a transplant or graft which is at risk of rejection can be treated with a composition of the invention in order to increase the number of cells, or activate the cells that attenuate the immune response that causes graft-versus-host disease in cases of transplantation.

As discussed, invention compositions can have anti-cell proliferative activity. Invention compositions are therefore useful for treating various disorders or physiological conditions characterized by abnormal or undesirable cell proliferation or abnormal or deficient cell differentiation.

Thus, in accordance with the invention, there are further provided methods for treating a subject with a composition of the invention having anti-cell proliferative activity. In one embodiment, a method includes administering to the subject a composition to inhibit cell proliferation. In one aspect, the subject has or is at risk of having a cell proliferative or differentiative disorder, or a physiological condition characterized by undesirable cell proliferation or abnormal differentiation. In another aspect, the subject is currently undergoing or will be undergoing a proliferative or differentiative disorder therapy (e.g., an anti-tumor therapy). In yet another aspect, the subject is administered with an invention composition prior to, contemporaneously with, or after administering a drug. In another embodiment, a method includes administering a composition to the subject to treat a solid tumor. In yet another embodiment, a method includes administering a composition to the subject in order to treat a liquid tumor. In various aspects, the subject having the tumor is administered with an invention composition prior to, contemporaneously with, or after another anti-tumor therapy.

As used herein, the term “proliferative disorder” means a pathological or non-pathological physiological condition characterized by aberrant cell proliferation or cell survival (e.g., due to deficient apoptosis). The term “differentiative disorder” means a pathological or non-pathological physiological condition characterized by aberrant or deficient cell differentiation. The term “solid tumor” refers to neoplasias or metastases that typically aggregate together and form a mass. Particular examples include visceral tumors such as gastric or colon cancer, hepatomas, venal carcinomas, lung and brain tumors/cancers. “Liquid tumor” refers to neoplasias of the haematopoetic system, such as lymphomas, myelomas and leukemias, or neoplasias that are diffuse in nature, as they do not typically form a solid mass. Particular examples of leukemias include acute and chronic exogeneous, acute and chronic lymphoblastic and multiple myeloma.

Proliferative or differentiative disorders or conditions amenable to treatment include diseases and physiological conditions, both benign and neoplastic, characterized by abnormal or undesirable cell numbers, cell growth or cell survival in a subject. Thus, such disorders or conditions may constitute a disease state and include all types of cancerous growths or oncogenic processes, metastatic tissues or malignantly transformed cells, tissues, or organs, or may be non-pathologic, i.e., a deviation from normal but which is not typically associated with disease, for example, tissue growth from wound repair that results in scarring.

Specific examples of such disorders include neoplasms or cancers, which can affect virtually any cell or tissue type, e.g., carcinoma, sarcoma, metastatic disorders or haematopoietic neoplastic disorders, e.g., leukemias. A metastatic tumor can arise from a multitude of primary tumor types, including but not limited to breast, lung, thyroid, head and neck, brain, lymphoid, gastrointestinal (mouth, esophagus, stomach, small intestine, colon, rectum), genito-urinary tract (uterus, ovary, cervix, bladder, testicle, prostate), kidney, pancreas, liver, bone, muscle, skin, etc.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from the cervix, lung, prostate, breast, head and neck, colon, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas refer to malignant tumors of mesenchymal cell origin. Exemplary sarcomas include for example, lymphosarcoma, liposarcoma, osteosarcoma, and fibrosarcoma.

Additional examples of proliferative disorders and conditions include haematopoietic neoplastic disorders. As used herein, “haematopoietic neoplastic disorder” includes diseases involving hyperplastic/neoplastic cells of haematopoietic origin, e.g., arising from myeloid, lymphoid or erythroid lineages, or precursor cells thereof. Typically, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (L. Vaickus, Crit Rev. in Oncol./Hemotol., 11:267-97 (1991)); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Anti-cell proliferative therapies including anti-tumor therapies include any treatment modality for the condition. For example, an anti-cell proliferative or anti-tumor treatment may comprise radiation treatment in which no drug is administered. The treatment may comprise administration of a chemical substance, such as a radioisotope, a drug, such as a chemotherapeutic agent, or a genetic therapy, such as an anti-oncogene (e.g., Rb, DCC, p53, etc.) or an antisense to an oncogene. Anti-cell proliferative and anti-tumor treatment modalities also include a tissue or organ transplant or graft, such as a bone marrow transplant.

The compositions of the invention can be administered in accordance with any protocol or route that achieves the desired effect. Thus, a pharmaceutical formulation can be administered once or more per day (e.g., at a low dose), or intermittently (e.g., every other day, once a week, etc. at a higher dose). The composition can be administered via inhalation, orally, intravenously, intravascularly, intraperitonealy, intramuscularly, subcutaneously, intracavity, transdermally, topically or by gradual perfusion over time or bolus infusion. Implantable devices, including microfabricated devices, for administering drugs are well known and are also applicable for delivering compositions of the invention to a subject.

A composition can be administered prophylactically to a subject prior to onset of immunosuppression, myclosuppression or anti-cell proliferative therapy. For example, a subject about to be treated with an immunosuppressing agent (e.g., a steroid) or an anti-cell proliferative therapy (e.g., radiation, chemotherapy, etc.) can be administered an invention composition in order to inhibit immunosuppression (e.g., myelosuppression) in the subject that occurs typically following treatment with the mmunosuppressing or anti-cell proliferative therapy.

The compositions also can be administered in association with any other treatment protocol. Other protocols include drug treatment, surgical resection, transplantation, radiotherapy, etc. The compositions can be administered prior to, contemporaneously with or following other treatment protocols.

Amounts administered, are typically in an “effective amount,” that is an amount sufficient to produce the desired affect. For example, where it is desired to increase the number of white blood cells in a subject, the effective amount will be that which detectably increases the number of white blood cells. Where it is desired to inhibit myelosuppression, the amount will be that which detectably increases the number of neutrophils, monocytes, macrophages, antigen-presenting cells, dendritic cells, Kupffer cells, Langerhans cells, microglial cells or mesangial cells. Similarly, where it is desired to increase antibody response, the amount will be that sufficient to increase HA and HL titres, for example. Where it is desired to inhibit cell proliferation, the amount will be sufficient to reduce target cell numbers, or prevent or reduce increases in numbers of target cells.

Where it is desired to treat a particular disorder, an effective amount will produce a reduction in the severity of the symptoms or progression of the disorder, which is a satisfactory clinical endpoint. For example, where the disorder comprises immunosuppression, a satisfactory endpoint is increased numbers of white blood cells, antibody titres or inhibiting decreases in monocyte cell numbers. Symptoms that are likely to improve following treatment with a composition of the invention include, for example, decreased susceptibility to bacteria, viral and fungal infection, and opportunistic infection. Where the disorder comprises a solid tumor, reducing tumor size, preventing further growth of the tumor, inhibiting proliferation of at least part of the tumor (e.g. 10% of the cells, or 20% or more) of the cells or inhibiting metastasis is a satisfactory clinical endpoint. Examination of a biopsied sample containing a liquid tumor (e.g., blood or tissue sample), can establish whether a reduction in numbers of tumor cells or inhibition of tumor cell proliferation has occurred. Alternatively, for a solid tumor, invasive and non-invasive imaging methods can ascertain a reduction in tumor size, or inhibiting increases in tumor size.

Decreasing counts of receptor of a receptor positive tumor, can be used to assess reduction or inhibition of proliferation. Amounts of hormone of a hormone producing tumor, e.g., breast cancer, can be used to assess reduction or inhibition of proliferation of a hormone producing tumor such as testicular, ovarian or breast carcinomas.

An effective amount of an invention composition that increases numbers of cells within the class of white blood cells for a human subject will typically range from about 500 to about 2500 mg/day, more likely between about 700 and 2000 mg/day. The skilled artisan will appreciate the various factors that may influence the dosage and timing required to treat a particular subject, including but not limited to the general health, age or gender of the subject, severity of the disease or disorder, previous treatments, clinical outcome desired and the presence of other diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” and “the” include plural referents unless the context clearly indicates otherwise. Thus, for example, reference to a “fraction” includes a plurality of fractions and reference to “the fraction” includes reference to one or more fractions.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLE I

This example describes a method for preparing a composition having immune stimulating or anti-tumor activity. The exemplified process utilizes material from Withania Somnifera to produce several fractions, denoted A, B and C, one or more of which, including one or more components within each of the fractions, have immune stimulating or anti-tumor activity. The fractions are combined and extracted with a solvent in order to produce a composition of the invention, referred to as WITHASOL having immunestimulating anti-tumor activity.

The steps for obtaining a plant extract of the invention from Withania Somnifera include cleaning the roots of Withania Somnifera to remove foreign matter; particulating the roots to obtain a particulated mass having particle size ranging from about 0.001 to about 10 mm3; contacting the particulate mass with a first medium polar solvent to obtain a first solution and a first residue; filtering the first solution from the first residue; evaporating the filtrate obtained from the first solution to remove the solvent and obtain a fraction denoted as fraction A. Examples of appropriate medium polar solvents include acetone, tetrahydrofuran and ethyacetate. The first residue is resuspended in a second polar solvent (e.g., 50% to 95% ethanol or about 40% to 60% isopropyl alcohol in water) for twelve to thirty-six hours to obtain a second solution and a second residue; filtering the second solution from the second residue to obtain a second filtrate; evaporating the second filtrate to remove its solvent to produce a fraction denoted fraction B. The second residue is re-suspended in a third solvent more polar than the second polar solvent to produce a third solution and a third residue. In one aspect, the third solvent comprises water contacting the second residue for twelve to thirty-six hours at temperatures ranging from ambient to 60° C. to obtain the third solution and third residue; clarifying the third solution to produce a third clarified solution; evaporating the third solvent to produce a fraction denoted fraction C. Fractions A, B and C are combined to produce an extract (total extract). Combined fractions A, B and C are then fractionated with a nonpolar solvent after making the solution aklaline to produce a composition having immune stimulating activity or anti-tumor activity. Buffers, solutions and solvents can additionally contain salts, chelating agents, preservatives and stabilizing agents (e.g., to stabilize pH within a particular range), which can be useful for removing impurities, increasing yield, or stabilizing or preventing degradation.

The production of Withania Somnifera total extract involves: Fraction A, extraction of plant with a medium polar solvent (e.g., acetone, tertahydrofuran, ethylacetate); followed by Fraction B, extraction of the first residue with a second polar solvent (e.g., isopropyl alcohol (IPA)-water (1:1), ethanol or methanol); and finally Fraction C, extraction of the second residue with a solvent more polar than the second solvent (e.g., hot water). The three fractions are combined. Typical physical characteristics of the composition (referred to as Withania somnifera total extract) are as follows:

pH of 5% aqueous suspension: 4.52

Weight per ml.: 1.2577 gm

Loss on drying at 900 C., under 15 mm Hg pressure for four Hrs.: 25.61% (by mass)

TLC pattern: Complies with the Working Standard. (The working standard is the first withasol preparation used successfully in pharmacological studies.)

For obtaining the composition of the invention, the combined fractions in paste form is treated with a non-polar solvent such as methylene chloride, ether, chloroform or the like made alkaline in order to convert alkaloids into their free bases, which are extractable with a non-polar solvent. Typically, the extract and the solvent are allowed to stand in a percolator for a period of 24 hours and the solute trickles down, is collected and is discarded. Trace solvent is removed from the residue by vacuum extraction or other suitable means. A solvent free residue is produced, referred to as WITHASOL, which is substantially or completely free of alkaloids or other components (e.g., withanolides, fats, protein, etc.). The composition need not be free of withanolides, fats, proteins or carbohydrates. Major portion of free withanolides, i.e. those not conjugated with glucose to form glyco-withanolides, are removed during extraction with a non-polar solvent.

Glyco-withanolide Assay

The lowest limit of concentration of actives for assay is 0.05%.

Withasol, 20 gms, weigh and mix with 200 ml 1M HCL, reflux 6 hours on boiling water bath and keep it overnight. Filter the mixture through whatman filter paper, and the filtrate is extracted with 75 ml of methylene chloride five times. Combined methylene chloride extract is washed twice with saturated sodium bicarbonate solution, followed by water, till the methylene chloride layer is free from acid. Then the methylene chloride layer is evaporated on rotary flash evaporator and the dried residue is mixed with 5 ml of 0.5M Methanolic Potassium Hydroxide and 25 ml methanol. This mixture is refluxed for 4 hours on water bath, cooled. Add 25 ml more methanol and titrate potentiometrically with 0.1M HCl. Also find out Blank reading, by taking 5 ml of 0.5M Methanolic Potassium Hydroxide and 50 ml methanol and titrating with 0.1M HCl.

$$\% Glyco - W = \frac{Blank Read . - Sample Read . \times Molarity of HCL \times 486 \times 100}{Weight of Sample in MG .}$$

Total Withanolide Assay

The lowest limit of concentration of actives for assay is 0.05%.

Withania somnifera root extract, 20 g, is exhaustively extracted with methylene chloride in soxhlet apparatus. Methylene chloride extract is fraction A. The residue mixed with 200 ml 1M HCl, refluxed 6 hours on boiling water bath and kept it overnight. Filter the mixture through Whatman filter paper, and the filtrate is extracted with 75 ml of methylene chloride five times, fraction B. mix fraction A and B and combined methylene chloride extract is washed twice with saturated sodium bicarbonate solution, followed by water, till the methylene chloride layer is free from acid. Then the methylene chloride layer is evaporated on rotary flash evaporator and the dried residue is mixed with 5 ml of 0.5M Methanolic Potassium Hydroxide and 25 ml methanol. This mixture is refluxed for 4 hours on water bath, cooled. Add 25 ml more methanol and titrate potentiometrically with 0.1M HCl. Also find out Blank reading, by taking 5 ml of 0.5M Methanolic Potassium Hydroxide and 50 ml methanol and titrating with 0.1M HCl.

$$\% Total W = \frac{Blank Read . - Sample Read . \times Molarity of HCL \times 486 \times 100}{Weight of Sample in MG .}$$

Batch 1 WITHASOL

Withania Somnifera total extract, 300 g (Kancor, B. No. Asw-003; fractions A,B and C.) was mixed with 25 ml water and 50 ml ammonia solution containing 25% NH40H; pH is ˜10 and extracted with 300 ml diethyl ether 6 times. The mixture was allowed to stand in a percolator for 24 hrs and the solute trickled down, is collected and the combined ether extract was discarded. The aqueous solution remaining after ether extraction was concentrated below 60° C. under reduced pressure to a consistency of a thick, viscous, dark red liquid. The yield of the product was 240 g (80%). Batch 1 was used in the subsequent animal pharmacology studies described herein.

Batch 2 WITHASOL

Withania Somnifera total extract, 300 gm was mixed with 50 ml water and 25 ml triethyl amine (pH˜10) and extracted with 500 ml methylene chloride. The mixture was stirred for 2 hrs and allowed to stand overnight. The lower layer of methylene chloride was withdrawn and discarded. The extraction was repeated twice. The aqueous solution remaining after removal of methylene chloride portion was concentrated below 60° C. under reduced pressure to a thick consistency of a viscous dark colored semisolid. The yield of the product was 222 gm (74%).

Batch 3 WITHASOL

Withania Somnifera total extract, 300 gm was mixed with 50 ml of a 25% ammonia solution (pH˜10) and extracted with 500 ml chloroform. The mixture was stirred for 2 hrs and allowed to stand overnight. The lower layer of chloroform was withdrawn and discarded. The extraction was repeated twice. The aqueous solution remaining after removal of chloroform was concentrated below 60° C. under reduced pressure to a thick consistency of a viscous dark colored semisolid. The yield of the product was 229.5 gm (76.5%).

TLC and HPLC Profiles

For TLC, about 1 g of sample is heated at 100° C. with 2N HCl for 3 h and extracted with methylene chloride. The Me2C12 extract was adjusted to 50 mg/ml and spotted on TLC plate with a hexane-methylene chloride-methanol (20:30:2) mobile phase. Spots were visualized by dipping the TLC plate in 1 %vanillin in phosphoric acid diluted 1:10 by methanol, followed by heating the plate at 105° C. for 15 min. The chromatogram was scanned using an Advanced American Biotechnology image scanner and associated software to convert the TLC spots into a gray scale image. The density of spots of each sample was measured along the axis to convert it into a peak pattern chromatogram; the area under the peak is proportional to the concentration of the constituent of the sample. This chromatogram is presented as a TLC profile of WITHASOL indicating the peak due to Withanolide-D (FIGS. 1A, 2A and 3A).

For HPLC, about 1 g of sample is heated at 100° C. with 2N HCl for 3 h and extracted with methylene chloride. The Me2Cl2 extract was evaporated to dryness and the residue was dissolved in HPLC grade methanol to prepare a final concentration of 1.5 mg/ml, which was injected (20:1) into the chromatograph. A methanol:water (60:40) mobile phase was used on a, RP C-18 column (Prochrome), 5, spherical, 250 mm×4.6 mm at a flow rate of 1.2 ml/min. Detector wavelength was 225 nm for the HPLC profile (FIGS. 1B, 2B and 3B).

The TLC profile of each WITHASOL batch 1, 2 and 3 was generally in accordance with the profiles shown in FIGS. 1A, 2A and 3A, respectively. Rf valves and percentage of the peaks for three batches are listed in Table 1. The TLC profile reveals 9 major peaks, one or more of which, or components therein, may have one or more of the activities of the invention compositions. The HPLC chromatogram of each WITHASOL batch 1, 2 and 3 was generally in accordance with the profiles shown in FIGS. 1B, 2B and 3B, respectively. Retention times of HPLC peaks are listed in Table 2.

### EXAMPLE II

This example describes the establishment and standardization of an animal model for measuring the immunostimulating activity, and anti-tumor activity, of WITHASOL. The animal model is a tumor bearing (sarcoma-180 ascitic tumor) and antigenically challenged BALB-c mouse strain.

Preparation of S-180 Cells for Implantation

Cells were grown in modified MEM (Earl's modification) at 37° C. in 5% CO₂ and the cell density was monitored from day 4 of incubation. Cells were harvested on day 7, by centrifugation at 2,000 rpm for 10 minutes and washed three times in sterile growth medium. The cell pellet was resuspended in sterile medium and cell density adjusted to 5.5×10⁶ cells/cc.

Animal Preparation

Male BALB/c mice (30), weighing and average of 25 gm (National Institute of Virology, Pune, India) were randomly divided into three equal groups, group I, group II and group III, and numbered with picric acid. Weight of each of the mice was recorded.

Antigen Preparation

Human ‘O’ group blood was collected aseptically in sterile Alsevar's solution (1:1 ratio). Red blood cells (RBCs) were separated by centrifugation at 3,000 rpm for 10 minutes and washed thrice in Alsevar's solution. Washed and packed RBCs were resuspended in Alsevar's solution and the cell density was adjusted to 8×10⁷ cells/cc.

Antigen Challenge and Tumor Implantation

The groups II and III mice were challenged with 0.5 cc of RBC suspension, intraperitoneally on day-1. Groups I and II mice were inoculated with 1.5×106 S-180 cells intraperitoneally on day 0.

Animal Inoculations Summarized

| | Group I | Group II | Group III |
|---|---|---|---|
| Tumor | + | + | − |
| Antigen | − | + | − |

Mice were weighed three times a week and any increase in weight was recorded. On day 22, one mouse from each group was sacrificed and autopsies were carried out. Visceral organs like liver, kidney, lung, spleen and nodular mass at site of inoculation was collected in fixative 10% formalin and sent for histopathological studies.

Tumor Sarcoma-180 was successfully implanted in BALB-c mice, revealing an increase in body weight, and a decrease in mean survival time. The increase in body weight was significant in Group I (P<0.05 and P<0.001) and also in Group II (P<0.01 and P<0.001) when compared to Group III, on day 6 and 13 respectively. In antigenically challenged mice (Group II) it was observed that tumor implantation was more significant as evidenced by a decrease in survival time, in comparison to mice receiving no antigenic challenge (Group I), (P<0.001). Mean survival time for Group II mice, 18.33 days, was less than Group I mice, 20.44 days, while Group III mice survived for more than 30 days. Autopsies of sacrificed mice revealed that tumor liquid mass was present only in peritoneal cavity as thick viscous fluid. Smears prepared from this fluid showed only sarcoma cells. In one animal a whitish gray nodular mass was present at the site of inoculation.

Histopathological studies revealed no infiltration or metastasis of tumor in organs like liver, spleen, kidneys and lungs, indicating ascitic nature of this tumor. Nodular mass was seen in some animals showing no significant weight change, which may be due to experimental variation in tumor implantation.

In sum, the increase in tumor mass in Groups I and II animals as compared to Group III animals produces a significant increase in body weight after day 6 of tumor implantation. This finding suggests that anti-tumor therapy can be started from about day 5 or day 6 onwards. The data also indicate that the S-180 tumor grows in antigenically challenged mice and antigenic challenge makes the mice more susceptible to tumor implantation and growth, resulting in decreased survival.

### EXAMPLE III

This example describes the establishment of a cyclophosphamide (CP) dose regimen and time course for treating tumors that causes immunosuppression in the antigenically challenged BALB/c mouse animal model implanted with ascitic sarcoma in order to determine optimal parameters for assessing WITHASOL efficacy.

Animal Treatment Groups

| Group^{a} | Dose (mg/Kg) | Route | Days of Administration |
|---|---|---|---|
| I | Vehicle (CMC) | po | 4 to 19 |
| II | 3.0 | po | 4 to 19 |
| III | Vehicle | ip | 7, 9, 11 |
| IV | 100 | ip | 7, 9, 11 |
| V | Vehicle | ip | 11 |
| VI | 150 | ip | 11 |

^{a}BALB/c mouse with antigen challenge and tumor inoculation used for all groups.

Cyclophosphamide (CP) Treatment of Tumor Bearing Animals

Step three, as described in Example II, was undertaken to establish a dose regimen of cyclophosphamide (CP), which decreases tumor growth in antigenically challenged BALB/c mouse with ascitic sarcoma. In brief, 60 male BALB/c mice, weighing an average of 25 g were randomly divided into six equal groups and numbered with picric acid and the weight of each recorded. Mice were antigenically challenged with 0.5 cc RBCs (1.44×10⁸ cells/cc) from human ‘O’ group blood in barbitone buffered saline prepared as described above. Mice from all groups were sensitized with the RBC suspension, intraperitoneally on day 1.

S-180 cells for animal implantation were prepared as described above. Since a large number of cells were required, cell culture was supplemented with fresh medium on day 6 and day 9 after the initial plating. Cells were harvested on day 11, as described above, resuspended in sterile medium and the cell density adjusted to 2.7×10⁶ cells /cc. Mice from all groups were inoculated with 0.5 cc S-180 cell suspension, intraperitoneally on day 0.

Treatment of Mice Groups

| Group^{a} | CP Dose (mg/Kg) | Route | Days of Administration |
|---|---|---|---|
| I | Vehicle (CMC) | po | 4 to 19 |
| II | 3.0 | po | 4 to 19 |
| III | Vehicle | ip | 7, 9, 11 |
| IV | 100 | ip | 7, 9, 11 |
| V | Vehicle | ip | 11 |
| VI | 150 | ip | 11 |

^{a}BALB-c mouse with antigen challenge and tumor inoculation used for all groups. Mice were weighed daily and weight of individual mouse was recorded.

Physiological Parameters

Body weight, survival time, hemoglobin content, red blood cell counts, total and differential white blood cells, platelets were all determined. On day 20, blood was collected from all mice in all groups in microcentrifuge tubes with EDTA as anticoagulant for blood cell counts. Smears of the blood samples were prepared and stained with Leishman's stain. Cell counts were taken on a Sysmex K-1000 cell counter (Toa Medical Instruments, Japan). As a counter check, two samples from each group were processed manually using an Improved Neubaeur's chamber.

The CP dose regimen was determined by comparing Groups I and II, Groups III and IV and Group V and VI using students t test. A significant decrease in weight of animals treated with CP (P<0.001) was observed for all three CP doses.

Comparison of groups I and II animals revealed no significant change in hemoglobin content, red cell counts, lymphocytes and platelet counts. Total white cell counts (P<0.05) and neutrophil counts (P<0.01) were significantly lowered while monocyte counts were significantly increased (P<0.05) in treated animals.

Comparison of Groups III and IV animals revealed no significant change in hemoglobin content, red cell counts, white cell total and differential counts and platelet counts.

Comparison of Groups V and VI animals revealed no significant change in hemoglobin content, red cell counts, platelet counts and monocytes. A significant increase in white cell counts (P<0.05) and neutrophils (P<0.01) was observed in CP treated animals. Results indicated that CP dose regime 150 mg/kg on day 11 i.p., produces maximum survival rate.

Kinetics of Cyclophosphamide Treatment

A study was then undertaken to determine the kinetics of cyclophosphamide treatment with respect to myelosuppression and immunosuppression. This step four aspect of the protocol establishes a time response curve for cyclophosphamide and determines optimal time for collection of blood samples and evaluation of hematological and serological parameters under the aforementioned conditions.

Animal Treatment Groups

| Group^{a} | Treatment | Blood Collection on Day |
|---|---|---|
| I | Vehicle (CMC) | Day 13 |
| II | CP (150 mg/Kg) | Day 13 |
| III | Vehicle | Day 15 |
| IV | CP | Day 15 |
| V | Vehicle | Day 17 |
| VI | CP | Day 17 |
| VII | Vehicle | Day 19 |
| VIII | CP | Day 19 |
| IX | Vehicle | Day 21 |
| X | CP | Day 21 |
| XI | Vehicle | Day 30 |
| XII | CP | Day 30 |

^{a}BALB-c mouse with antigen challenge and tumor inoculation used for all groups. On day 11 six groups received vehicle (CMC 2%) while other six groups received ip cyclophosphamide at 150 mg/Kg.

In brief, 120 male BALB/-c mice, weighing an average of 25 g strain were divided into twelve equal groups, numbered with picric acid and their weight was recorded.

Mice were antigenically challenged with 0.5 cc of RBC suspension (1.0×10⁸ cells/cc), intraperitoneally on day 1 as previously described. Mice from all groups were sensitized with RBC and a challenge dose was given on day 10.

S-180 cells for animal implantation were prepared as previously described. Since a large number of cells were required, the cell culture was supplemented with fresh medium on day 5 and day 8 after the initial plating. Cells were harvested on day 11, as previously described, resuspended in sterile medium and the cell density adjusted to 1.6×10⁶/cc. Mice from all groups were inoculated with 0.5 cc S-180 cell suspension, intraperitoneally on day 0.

Treatment of Mice Groups

| Group^{a} | Treatment | Blood Collection on Day |
|---|---|---|
| I | Vehicle (CMC) | Day 13 |
| II | CP (150 mg/Kg) | Day 13 |
| III | Vehicle | Day 15 |
| IV | CP | Day 15 |
| V | Vehicle | Day 17 |
| VI | CP | Day 17 |
| VII | Vehicle | Day 19 |
| VIII | CP | Day 19 |
| IX | Vehicle | Day 21 |
| X | CP | Day 21 |
| XI | Vehicle | Day 30 |
| XII | CP | Day 30 |

^{a}BALB-c mouse with antigen challenge and tumor inoculation used for all groups. On day 11 six groups received vehicle (CMC 2%) while other six groups received ip cyclophosphamide at 150 mg/Kg. Mice were weighed daily and weight was recorded.

Physiological Parameters

Body weight, survival time, hemoglobin content, red blood cell counts, total and differential white blood cells, platelets and hemagglutinating and hemolytic antibody titres were determined. Blood was collected from all mice in all groups as per schedule in microcentrifuge tubes with EDTA as anticoagulant for blood cell counts. Smears of the blood samples were prepared and stained with Leishman's stain. Cell counts were taken on a Sysmex K-1000 cell counter (Toa Medical Instruments, Japan) standardized using mice blood (collected from healthy, normal, adult mice), under the guidance of service engineer. Antibody titres were estimated using 96 well, U-bottom microtiter plates, double diluting serum samples and using 10% Human RBC suspension and complement (guinea pig serum).

Available animals were distributed in twelve equal groups in such a way that variation in weight of animals between and within groups was not significant. Tumor implantation and growth followed in the same manner as in previous studies, although variation in weight within the animal groups was large due to variations in the initial weights/age of the animals.

A single cyclophosphamide dose of 150 mg/Kg, on day 11, resulted in regression of tumor in respective groups. Tumor relapse was observed after a few days of cyclophosphamide treatment, and the animals died from the tumor regrowth, as evidenced by weight increase.

The single does of cyclophosphamide treatment resulted in a significant decrease in hemoglobin content (P<0.01) on day 17; red blood cell counts increased (P<0.09) on day 19; and platelet counts were unaffected. White blood cell counts were significantly decreased, on day 13 (P<0.01) and on day 15 (P<0.0000006). These observations are consistent with previous studies that cyclophosphamide acts as a myelosuppressive agent. While restoring from myclosuppression, differential counts show a large variability, initial lymphocytosis followed by polymorpholeucocytosis; polymorphonuclear leukocytosis is an initial increase in lymphocyte counts, followed by an increase in polymorphs.

Immunosuppressive effect of cyclophosphamide was also evidenced by a decrease in hemolytic antibody titers (complement fixing-CF) on day 13 (P<0.00006), day 15 (P<0.001), day 17 (P<0.001), day 19 (P<0.01) and day 21 (P<0.0003). A decrease in hemagglutinating antibody (HA) titers was also observed on day 13 (P<0.004), day 15 (P<0.00003), day 17 (P<0.000000003), day 19 (P<0.001) and day 21 (P<0.0007).

This study indicates that the maximum myelosuppressive and immunosuppressive effect of cyclophosphamide was achieved on day 15, or in between day 15 and day 17. Immune modulatory effects of WITHASOL in tumor bearing and cyclophosphamide treated animals can therefore be studied in blood collected from between days 15 and 17.

### EXAMPLE IV

This example describes double-blind studies of the immunostimulatory activity of WITHASOL and other herbal drug preparations in cyclophosphamide (CP) treated antigenically challenged BALB/c mouse animals implanted with ascitic sarcoma (S-180).

As described herein, treatment of ascitic sarcoma-180 bearing mice with cyclophosphamide results in marked regression of tumor growth but is accompanied with undesirable side effects such as myelosuppression and immunosuppression. To evaluate immunomodulatory activity of different herbal drug preparations in the mice ascitic sarcoma-180 model and to determine whether the drugs have any anti-tumor activity per se, double-blind studies of the CP treated S-180 implanted animals administered various herbal preparations were undertaken.

Nine test drugs in doses of 100 mg/kg body wt each assigned confidential codes L, M, N, O, P, Q, R, S and T were prepared in 2.0% sterile CMC solution as 25 mg/ml solution (the codes were not known to the investigator at the time of the studies; Drug ‘R’ was WITHASOL).

In brief, 140 male BALB/c mice, weighing an average of 25 g were randomly divided into 14 equal groups, numbered with picric acid and the weight of each recorded. Mice were antigenically challenged with 0.5 cc human ‘O’ group blood RBCs (1.4×10⁸ cells/cc) in barbitone buffered saline prepared as described above. Mice from all groups were sensitized intraperitoneally (ip) with the RBC suspension on day 1. A challenge dose was administered on day 10.

S-180 cells for animal implantation were prepared as described above. Cells were grown, harvested, resuspended in sterile medium and the cell density adjusted to 2.1×10⁶ cells/cc. Mice from all groups were inoculated with 0.5 cc S-180 cell suspension, intraperitoneally on day 0.

Mice groups were treated with drugs under study from day 1 to day 15, per oral route. Control mice received vehicle only, 2.0% CMC.

Treatment Of Mice Groups

| | CP Treatment | Oral Treatment |
|---|---|---|
| Group^{a} i.p. | on day 11 | from day 1 to 15 |
| I | Vehicle (CMC) | CMC |
| II | CP (150 mg/Kg) | CMC |
| III | Vehicle | CMC |
| IV | CP | CMC |
| V | CP | L |
| VI | CP | M |
| VII | CP | N |
| VIII | CP | O |
| IX | CP | P |
| X | CP | Q |
| XI | CP | R |
| XII | cP | S |
| XIII | CP | Mesna |
| XIV | CP | T |

^{a}BALB-c mouse with antigen challenge and tumor inoculation used for all groups. On day 11, mice in respective groups received vehicle (CMC 2%), while other groups received ip cyclophosphamide at 150 mg/Kg. Mice were weighed daily and the weight was recorded. Each group was stabilized at 6 animals.

Physiological Parameters

Body weight, survival time, hemoglobin content, cell counts for red blood cells, total and differential white blood cells, platelets and hemagglutinating and hemolytic antibody titres were determined. Blood was collected from all mice in all groups on day 16 in microcentrifuge tubes with EDTA as anticoagulant for blood cell counts and without anticoagulant for serum separation. Smears of the blood samples were prepared and stained as previously described. Cell counts were taken on a Sysmex K-1000 cell counter (Toa Medical Instruments, Japan) standardized as previously described. Antibody titres were estimated using 96 well, U-bottom microtitre plates, double diluting serum samples and using 5% Human RBC suspension and complement (guinea pig serum). Data was collected and analyzed by ANOVA single factor analysis (alpha =0.05) tool in MS Office Excel™ (Version 5.0).

Hematological and Serological
(Mean +/− Std. Dev.)

| | Difference | | RBC | | WBC Total |
|---|---|---|---|---|---|
| | in Weight (g) | Hb | Count | Platelet Count | Count |
| | from Day 1 to 16 | (g/dl) | (×10⁶/mcl) | (×10³/mcl) | (×10³/mcl) |
| Group I | 1.17 +/− 0.753 | 14.48 +/− 0.866 | 5.12 +/− 0.412 | 973.67 +/− 207.969 | 8.2 +/− 1.495 |
| Tumor −, CP −, Vehicle | | | | | |
| Group II | −0.5 +/− 0.837 | 13.8 +/− 1.103 | 4.37 +/− 0.718 | 966.33 +/− 324.879 | 3.8 +/− 1.226 |
| Tumor −, CP +, Vehicle | | | | | |
| Group III | 4.67 +/− 3.386 | 13.43 +/− 2.439 | 4.20 +/− 0.976 | 992.83 +/− 231.575 | 11.3 +/− 2.897 |
| Tumor +, CP −, Vehicle | | | | | |
| Group IV | −0.83 +/− 2.563 | 11.07 +/− 3.812 | 3.54 +/− 1.332 | 614.17 +/− 287.466 | 4.53 +/− 0.797 |
| Tumor +, CP +, Vehicle | | | | | |
| Group V | 2.67 +/− 2.251 | 12.12 +/− 2.252 | 3.81 +/− 0.727 | 884 +/− 180.882 | 5.98 +/− 0.935 |
| Tumor +, CP + Drug L | | | | | |
| Group VI | 1.17 +/− 3.601 | 14.02 +/− 1.963 | 4.30 +/− 0.825 | 1117.33 +/− 225.240 | 6.37 +/− 1.727 |
| Tumor +, CP +, Drug M | | | | | |
| Group VII | 3.33 +/− 2.582 | 9.98 +/− 2.230 | 2.74 +/− 0.67 | 697.67 +/− 335.109 | 6.35 +/− 1.422 |
| Tumor +, CP +, Drug N | | | | | |
| Group VIII | 0.33 +/− 3.141 | 12.27 +/− 3.008 | 3.48 +/− 1.009 | 722.67 +/− 404.687 | 5.7 + 1.137 |
| Tumor +, CP +, Drug O | | | | | |
| Group IX | 1.33 +/− 2.733 | 12.78 +/− 1.579 | 3.94 +/− 0.513 | 793.83 +/− 371.637 | 6.23 + 1.830 |
| Tumor +, CP +, Drug P | | | | | |
| Group X | −0.1 +/− 1.414 | 14.32 +/− 1.219 | 4.24 +/− 0.429 | 591.83 +/− 256.422 | 6.07 +/− 0.641 |
| Tumor +, CP +, Drug Q | | | | | |
| Group XI | 0.17 +/− 1.835 | 12.62 +/− 2.272 | 3.38 +/− 0.728 | 656.5 +/− 130.698 | 6.17 +/− 0.709 |
| Tumor +, CP +, Drug R | | | | | |
| Group XII | −0.5 +/− 1.871 | 14.03 +/− 1.540 | 3.80 +/− 0.566 | 636.17 +/− 137.520 | 4.5 +/− 0.548 |
| Tumor +, CP +, Drug S | | | | | |
| Group XIII | −2.5 +/− 1.049 | 14.08 +/− 1.689 | 4.12 +/− 0.609 | 608.83 +/− 239.813 | 3.67 +/− 1.218 |
| Tumor +, CP +, Mesna | | | | | |
| Group XIV | −2.67 +/− 1.751 | 15.37 +/− 1.481 | 4.25 +/− 0.661 | 822.67 +/− 234.142 | 7.13 +/− 0.949 |
| Tumor +, CP +, Drug T | | | | | |

| | WBC Difference Count (%) | | HA Titre | CF Titre |
|---|---|---|---|---|
| | Polymorph | Lymphocytes | (Log₂) | (Log₂) |
| Group I | 26.68 +/− 6.861 | 73.32 +/− 6.861 | 6.83 +/− 0.408 | 6.17 +/− 0.408 |
| Tumor −, CP −, Vehicle | | | | |
| Group II | 36.2 +/− 2.7897 | 63.73 +/− 2.7897 | 1.5 +/− 5.48 | 1.00 |
| Tumor −, CP +, Vehicle | | | | |
| Group III | 55.98 +/− 11.425 | 44.02 +/− 11.425 | 5.5 +/− 0.548 | 4.5 +/− 0.5 [symbol] |
| Tumor +, CP −, Vehicle | | | | |
| Group IV | 41.42 +/− 8.634 | 58.18 +/− 8.634 | 2.17 +/− 0.837 | 1.83 +/− 0.408 |
| Tumor +, CP +, Vehicle | | | | |
| Group V | 48.68 +/− 9.550 | 51.32 +/− 9.550 | 1.50 +/− 0.548 | 1.83 +/− 0.408 |
| Tumor +, CP + Drug L | | | | |
| Group VI | 51.73 +/− 6.105 | 48.27 +/− 6.105 | 4.83 +/− 0.753 | 4.83 +/− 0.408 |
| Tumor +, CP +, Drug M | | | | |
| Group VII | 55.52 +/− 8.972 | 44.48 +/− 8.972 | 5.5 +/− 0.548 | 5.83 +/− 0.408 |
| Tumor +, CP +, Drug N | | | | |
| Group VIII | 39.68 +/− 10.861 | 60.32 +/− 10.861 | 5 +/− 0.894 | 4.33 +/− 0.516 |
| Tumor +, CP +, Drug O | | | | |
| Group IX | 46.4 +/− 17.810 | 53.6 +/− 17.810 | 3.17 +/− 0.753 | 3.33 +/− 0.516 |
| Tumor +, CP +, Drug P | | | | |
| Group X | 32 +/− 4 | 68 +/− 4 | 5.17 +/− 0.753 | 5.83 +/− 0.408 |
| Tumor +, CP +, Drug Q | | | | |
| Group XI | 40.47 +/− 11.591 | 59.53 +/− 11.591 | 5 +/− 0.632 | 5.17 +/− 0.753 |
| Tumor +, CP +, Drug R | | | | |
| Group XII | 39.55 +/− 12.095 | 60.45 +/− 12.095 | 6.17 +/− 0.753 | 5.17 +/− 0.408 |
| Tumor +, CP +, Drug S | | | | |
| Group XIII | 37.58 +/− 9.902 | 62.42 +/− 9.902 | 2.17 +/− 0.408 | 1.83 +/− 0.4 [symbol] |
| Tumor +, CP +, Mesna | | | | |
| Group XIV | 36.70 +/− 6.266 | 63.30 +/− 6.266 | 5.33 +/− 0.516 | 5.67 +/− 0.516 |
| Tumor +, CP +, Drug T | | | | |

Comparison of groups I and II to determine the effect of CP in tumor free animals revealed a decrease in body weights (P=0.005). No significant change in hemoglobin content or in platelet counts. Red cell counts lowered (P=0.05). White cell total counts are lowered (P=0.0002). Increased polymorph percent (P=0.01) accompanied with decreased lymphocyte percent (P=0.01) also was observed. Both HA antibody titers (P=0.0000000003) and HL antibody titers (P=0.000000000001) decreased. The results conform with earlier findings that CP is a myelo and immunosuppressive agent.

Comparison of groups I and III to determine the effect of tumor on body weight in the absence of CP treatment revealed increased body weight (P=0.03). No significant change in hemoglobin content, red cell counts, and platelet counts was detected. Increased white cell total counts (P=0.04) and a significant increase in polymorph percent (P=0.0003), accompanied with decrease in lymphocyte percent was also observed. Both HA antibody titers (P=0.0007) and HL antibody titers (P=0.0001) decreased. These results conform with earlier findings that tumor increases body weight, white cell total counts, and hemagglutinating antibody titres.

Comparison of groups III and IV to determine the effect of CP in tumor-bearing animals revealed decreased body weight (P=0.01). No significant change in hemoglobin content and red cell counts was detected. Decreased patelet counts (P=0.03), white counts (P=0.0003), polymorph percentage (P=0.04) and increase lymphocyte percentage (P=0.04) was also observed. Both HA antibody titers (P=0.00002) and HL antibody titers (P=0.0000) decreased. The results conform with earlier findings that CP as an antitumor agent decreases body weight and causes severe leuopenia and immunosuppression.

Comparison of groups IV and V to determine the protective effect of drug L in tumor-bearing CP-treated animals revealed a significant increase in body weight (P=0.03). No significant change in hemoglobin content, red cell counts, and platelet counts was detected. Increased white cell total counts (P=0.02) was observed but there was no significant change in HL antibody titers. These results indicate that drug L has not effect on body weight, counteractive effect on CP-induced myelosuppression and, at the same time, may have potentiating effect on CP-induced immuosuppression (i.e., causes a further drop in antibody titers than CP alone).

Comparison of groups IV and VI to determine the protective effect of drug M in tumor-bearing CP-treated animals revealed no significant change in body weight, hemoglobin content, or red cell counts. Both platelet counts (P=0.007) and white cell total counts (P=0.04) increased significantly. Polymorph percent (P=0.04) significant increased which was accompanied with decreased lymphocyte percent (P=0.04). Both HA antibody titers (P=0.005) and HL antibody titers (P=0.0000005) significantly increased. These results indicate that drug M has protective effect towards CP-induced thrombocytopenia and leucopenia. Drug M also has immunostimulatory activity although increased polymorph percent may indicate an inflammatory reaction.

Comparison of groups IV and VII to determine the protective effect of drug n in tumor-bearing CP-treated animals revealed a significant increase in body weight (P=0.02). No significant change in hemoglobin content, red cell counts, and platelet counts was detected. Both white cell total counts (P=0.02) and polymorph percent (P=0.02) significantly increased which was accompanied with decreased lymphocyte percent (P=0.02). Both HA antibody titers (P=0.00002) and HL antibody titers (P=0.00000002) significant increased. These results indicate that drug N has no effect on body weight, and a marginal protective effect on CP-induced myelosuppression and immunosuppression.

Comparison of groups IV and VIII to determine the protective effect of drug 0 in tumor-bearing CP-treated animals revealed no significant change in body weight, hemoglobin content, red cell counts, platelet counts, and white cell total and differential counts. Both HA antibody titers (P=0.005) and HL antibody titers (P=0.001) significantly increased. These results indicate that drug O has immunostimulatory activity but has no protective effect on tumor and CP-induced myelosuppression.

Comparison of groups IV and IX to determine the protective effect of drug P in tumor-bearing CP-treated animals revealed no significant change in body weight, hemoglobin content, red cell counts, platelet counts, white cell total and differential counts, and HA antibody titers. A significant increase in HL antibody titers (P=0.02) was observed. These results indicate that drug P has no protective effect on body weight, CP-induced myelo and immunosuppression.

Comparison of groups IV and X to determine the protective effect of drug Q in tumor-bearing CP-treated animals revealed no significant change in body weight, hemoglobin content, red cell counts, and platelet counts. white cell total counts (P=0.004) significantly increased. Polymorph content (P=0.03) significantly decreased which was accompanied with increased lymphocyte percent (P=0.03). Both HA antibody titers (P=0.0002) and HL antibody titers (P=0.00000002) significant increased. These results indicate that drug Q has no effect on body weight, but has a protective effect towards CP-induced myelo and immunosuppression. Drug Q may also have anti-inflammatory activity (lowered polymorphs).

Comparison of groups IV and XI to determine the protective effect of drug R in tumor-bearing CP-treated animals no significant change in body weight, hemoglobin content, red cell counts, and platelet counts. White cell total counts (P=0.004) significant increased and there was no significant change in differential counts. Both HA antibody titers (P=0.0002) and HL antibody titers (P=0.000006) significant increased. These results indicate that drug R has no effect on body weight, but has a protective effect on CP-induced myclo and immunosuppression.

Comparison of groups IV and XII to determine the protective effect of drug S in tumor-bearing CP-treated animals revealed no significant change in body weight, hemoglobin content, red cell counts, platelet counts, and white cell total and differential counts. Both HA antibody titers (P=0.00001) and HL antibody titers (P=0.0000002) significantly increased. These results indicate that drug S has immunostimulatory activity but has no effect on body weight and CP-induced myelosuppression.

Comparison of groups IV and XIII to determine the protective effect of Mesna in tumor-bearing CP-treated animals revealed no significant change in body weight, hemoglobin content, red cell counts, platelet counts, white cell total and differential counts, and HA and HL antibody titers. These results indicate that drug Mesna has no effect on body weight and CP-induced myelo and immunosuppression.

Comparison of groups IV and XIV to determine the protective effect of drug T in tumor-bearing CP-treated animals revealed no significant change in body weight. Hemoglobin content (P=0.03) significantly increased. No significant change in red cell counts, and platelet counts was detected. White cell total counts significantly increased (P=0.0005) but there was no significant change in differential counts. Both HA antibody titers (P=0.00004) and HL antibody titers (P=0.0000001) and significantly increased. These results indicate that drug T has no effect on body weight, protective effect on CP-induced myelo and immunosuppression. Drug T also has mild hematinic activity.

Thus, these results indicate that drug ‘R’ (WITHASOL) reverses cyclophosphamide-induced immunosuppression. Although WITHASOL does not appear to have a significant effect on hemoblogin content, platelet counts, white blood cell counts and antibody titers in untreated tumor bearing animals this drug significantly causes tumor regression and reverses immunosuppression (including myelosuppression) in cyclophosphamide treated tumor bearing animals.

Drugs L, M, N, O, P, Q, R, S and T are identified as follows:

L=Tinospora Satwa extract, Low dose

M=Asparagus, Non-polar extract, Low dose

N=Asparagus, Polar extract, Low dose

O=Tinospora, Non-polar extract, Low dose

P=Tinospora, water extract, Low dose

Q=Withania extract/Tinospora extract, Low dose

R=WITHASOL

S=Withania extract, Non-polar, Alkaloid free, Low dose

T=Vitamin C

In sum, the aforementioned studies indicate that:

(a) tumor could grow in BALB-c mice and antigen challenge increases lethality of the tumor to a small extent;

(b) WITHASOL stimulates immune responsiveness, as illustrated by increased numbers of white cell total counts, monocytes and HA and HL titers;

(c) WITHASOL significantly affects hematological and immunological parameters in selected strain of mice;

(d) the cyclophosphamide dose regimen effects tumor regression without inducing excess toxicity and lethality, a dose of 150 mg/Kg given ip on day 11, in antigenically challenged and ascitic sarcoma bearing mice, produces greatest survival rate; and

(e) an appropriate time for blood collection for evaluation of hematological and serological parameters under the experimental conditions is about day 16 after tumor implantation.

(f) Withasol does not counteract or interfere with tumor killing properties of cyclophosphamide.

### EXAMPLE V

This example describes dosing studies of WITHASOL in cyclophosphamide (CP) treated antigenically challenged BALB/c mouse animals implanted with ascitic sarcoma. This example further demonstrates the immunostimulatory, anti- myelosuppressive and anti-tumor potentiation activity of WITHASOL in animals.

Following the established protocol, tumor was implanted on day 0. Test drugs as suspensions in 2.0% sterile CMC (vehicle) were prepared by Bio-Ved Laboratories. Antigenically sensitized (day 1) and challenged (day 10) mice, received test substances from day 1 to day 15 per oral route. Animals received appropriate vehicle (2.0% CMC) or CP (150 mg/kg) i.p. on day 11 and blood was collected on day 16. Body weights and gross observations were monitored during treatment.

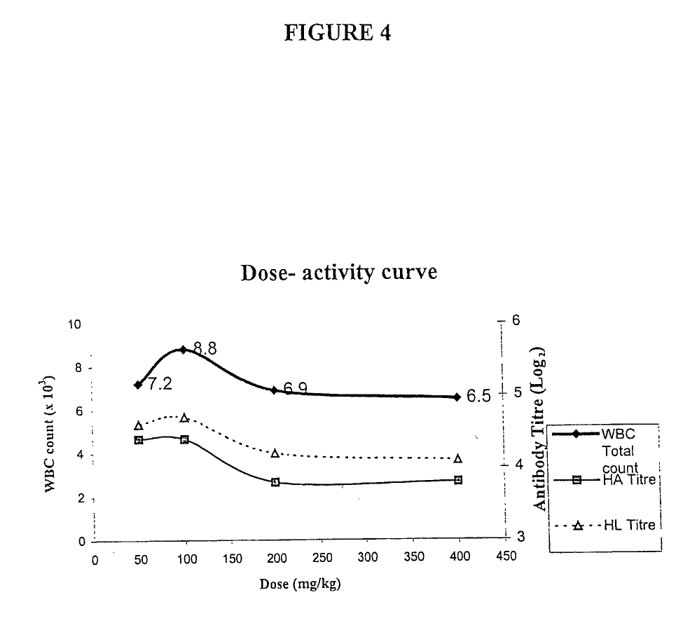

Four doses, 400 mg/kg, 200 mg/kg, 100 mg/kg and 50 mg/kg of WITHASOL coded as R-1, R-2, R-3 and R-4, respectively, were used for this study. Decoding was done after the study was completed. The dose-activity relationship is shown in FIG. 4.

Treatment Groups

| Group^{a} | Tumor | CP | Drug/Vehicle |
|---|---|---|---|
| I | − | − | Vehicle |
| II | − | + | Vehicle |
| III | + | − | Vehicle |
| IV | + | + | Vehicle |
| V | + | + | R-1 |
| VI | + | + | R-2 |
| VII | + | + | R-3 |
| VIII | + | + | R-4 |

^{a}Animals from Group V, VI, VII and VIII animals were active, with healthy fur coats as compared with Group II, III and IV animals.

Survival on Day 16 (Day of Bleeding)

| Group | No. of Animals Survived | Comments |
|---|---|---|
| I | 10 | — |
| II | 10 | — |
| III | 6 | Due to tumor as evident from |
| | | general observations. |
| IV | 8 | Due to tumor as evident from |
| | | general observations. |
| V | 10 | — |
| VI | 10 | — |
| VII | 10 | — |
| VIII | 9 | Due to tumor as evident from |
| | | general observations. |

Data analysis was carried out using ANOVA single factor @<0.05 for comparison of the groups. The data is summarized in the Table as follows:

Hematological & Serological Parameters
(Mean +/− Std. Dev.)

| | Difference in | | RBC | Platelet | WBC Total |
|---|---|---|---|---|---|
| | Weight (g) (from | Hb | Count | Count | Count |
| | Day 1 to 16) | (g/dl) | (×10⁶/mcl) | (×10³mcl) | (×10³/mcl) |
| Group I | 1.6 +/− 1.07 | 15.0 +/− 0.08 | 5.01 +/− 0.53 | 993.8 +/− 133.89 | 8.3 +/− 1.52 |
| Tumor −, CP −, Vehicle | | | | | |
| Group II | 0.4 +/− 0.52 | 12.9 +/− 1.40 | 4.04 +/− 0.54 | 1170.2 +/− 152.33 | 3.6 +/− 0.67 |
| Tumor −, CP +, Vehicle | | | | | |
| Group III | 10.7 +/− 3.72 | 12.3 +/− 2.60 | 3.65 +/− 1.08 | 805.5 +/− 300.13 | 15.2 +/− 4.15 |
| Tumor +, CP −, Vehicle | | | | | |
| Group IV | 4.9 +/− 2.36 | 12.8 +/− 1.03 | 3.79 +/− 0.46 | 906.0 +/− 312.76 | 6.2 +/− 2.17 |
| Tumor +, CP +, Vehicle | | | | | |
| Group V | 4.5 +/− 2.92 | 13.2 +/− 1.17 | 4.30 +/− 0.59 | 890.3 +/− 245.42 | 6.5 +/− 1.96 |
| Tumor +, CP + Drug R-1 | | | | | |
| Group VI | 2.5 +/− 2.64 | 13.7 +/− 1.11 | 4.3 +/− 0.76 | 837.1 +/− 304.22 | 6.9 +/− 3.10 |
| Tumor +, CP +, Drug R-2 | | | | | |
| Group VII | 4.4 +/− 2.67 | 12.1 +/− 1.97 | 3.93 +/− 0.49 | 912.8 +/− 253.42 | 8.8 +/− 2.15 |
| Tumor +, CP +, Drug R-3 | | | | | |
| Group VIII | 1.2 +/− 2.11 | 12.1 +/− 1.64 | 4.38 +/− 0.48 | 878.2 +/− 341.96 | 7.2 +/− 2.44 |
| Tumor +, CP +, Drug R-4 | | | | | |

| | WBC Difference Count (%) | | HA Titre | CF Titre |
|---|---|---|---|---|
| | Polymorph | Lymphocytes | (Log₂) | (Log₂) |
| Group I | 22.7 +/− 1.52 | 77.3 +/− 1.52 | 7.5 +/− 0.71 | 7.1 +/− 0.99 |
| Tumor −, CP −, Vehicle | | | | |
| Group II | 23.3 +/− 4.35 | 76.7 +/− 4.35 | 4.4 +/− 0.52 | 4.0 +/− 0.82 |
| Tumor −, CP +, Vehicle | | | | |
| Group III | 67.9 +/− 4.08 | 32.1 +/− 4.08 | 6.2 +/− 0.75 | 5.2 +/− 0. [symbol] |
| Tumor +, CP −, Vehicle | | | | |
| Group IV | 58.1 +/− 8.70 | 4.19 +/− 8.70 | 3.3 +/− 0.89 | 3.5 +/− 0.53 |
| Tumor +, CP +, Vehicle | | | | |
| Group V | 53.1 +/− 11.94 | 46.9 +/− 11.94 | 3.8 +/− 0.92 | 4.1 +/− 0.57 |
| Tumor +, CP + Drug R-1 | | | | |
| Group VI | 54.4 +/− 11.70 | 45.6 +/− 11.70 | 3.8 +/− 0.79 | 4.2 +/− 0.63 |
| Tumor +, CP +, Drug R-2 | | | | |
| Group VII | 58.2 +/− 10.20 | 41.9 +/− 10.20 | 4.4 +/− 0.52 | 4.7 +/− 0.48 |
| Tumor +, CP +, Drug R-3 | | | | |
| Group VIII | 56.7 +/− 14.21 | 43.3 +/− 14.21 | 4.4 +/− 0.73 | 4.6 +/− 0.73 |
| Tumor +, CP +, Drug R-4 | | | | |

A comparison of groups I and II to determine the effect of CP in tumor-free animals revealed decreased body weights (P=0.005); decreased hemoglobin content (P=0.0008); decreased red cell counts (P=0.0008); and increased platelet counts (P=0.01). White cell total counts decreased (P=0.00000005) and there was no significant change in white cell differential counts. Both HA antibody titres (P=0.000000002) and HL antibody titres (P=0.0000005) decreased. This data confirms CP as myelo and immunosuppressive agent.

A comparison of groups I and III animals to determine the effect of tumor in absence of CP treatment revealed increased body weights (P=0.000004), decreased hemoglobin content (P=0.01) and decreased red cell counts (P=0.004). There was no significant change in platelet counts. White cell total counts increased (P=0.0003) as did polymorph percent (P=0.000000000002), which was accompanied by decreased lymphocyte percent. Both HA antibody titres (P=0.003) and HL antibody titres (P=0.002) decreased. This data confirms that tumor increases body weights, decreases hemoglobin content, red cell counts and antibody titres and increases white cell total counts with severe polymorphonuclear leucocytosis.

A comparison of groups III and IV animals to determine the effect of CP in tumor-bearing animals revealed decreased body weights (P=0.004) and no significant change in hemoglobin content, red cell counts or platelet counts. There was a decrease in white cell total counts (P=0.0002) and polymorph percentage (P=0.03) accompanied with increased lymphocyte percentage. Both HA antibody titres (P=0.00003) and HL antibody titres (P=0.002) decreased. This data confirms that CP anti-tumor agent causes severe leucopenia and immunosuppression.

Code Profiles

A comparison of groups IV and V to determine the protective effect of R-1 in tumor-bearing CP treated animals revealed no significant change in body weight, hemoglobin content, red cell counts, platelet counts, white cell total and differential counts, and HA antibody titres. There was an increase in HL antibody titres (P=0.04). R-1 had no statistically significant counteractive activity on CP induced myelosuppression and immunosuppression, except to increase hemolytic antibody titres.

A comparison of groups IV and VI to determine the protective effect of R-2 in tumor-bearing CP treated animals revealed no significant change in body weight, hemoglobin content, red cell counts, platelet counts, white cell total and differential counts, and HA antibody titres. There was an increase in HL antibody titres (P=0.02). R-2 had no statistically significant counteractive activity on CP induced myclosuppression and immunosuppression, except to increase hemolytic antibody titres.

A comparison of groups IV and VII to determine the protective effect of R-3 in tumor-bearing CP treated animals revealed no significant change body weight, hemoglobin content, red cell counts or platelet counts. A significant increase in white cell total counts (P=0.02) was observed but there was no significant change in white cell differential counts. White blood cell differential count is the breakdown in percentage of the different blood cells e.g. neutrophils, lympocytes, basophils, eosinophils, monocytes and bands. Both HA antibody titres (P=0.003) and HL antibody titres (P=0.0001) increased significantly. R-3 had no effect on body weight but had a statistically significant protective effect against CP induced myclosuppression and immunosuppression.

A comparison of groups IV and VIII to determine the protective effect of R-4 in tumor-bearing CP treated animals revealed a significant decrease in body weights (P=0.004). There was no significant change in hemoglobin content, platelet counts, white cell total or differential counts, but red cell counts increased (P=0.02). Both HA antibody titres (P=0.008) and HL antibody titres (P=0.004) increased significantly. R-4 decreased body weights and increased red cell counts, had no statistically significant protective effect on CP induced myclosuppression but has an immunostimulatory activity.

In sum, drug codes R-1 and R-2 had no effect on body weights and did not exhibit protection towards CP induced myelosuppression but immunostimulation with respect to hemolytic antibody titres is present. R-3 had no effect on body weights and protects the animals from both CP induced myelosuppression and immunosuppression, as evident from increased white cell total counts and antibody titres. R-4 potentiates anti-tumor activity of CP as evidenced by decreased body weights. R-4 appears to have hematinic properties as evidenced by increased red cell counts. Drug R-4 also has immunostimulatory activity but did not appear to exhibit protection against CP induced myelosuppression.

Based on these data, R-3 can be selected as the optimum dose of WITHASOL for protection against CP induced myelo and immunosuppression. R-4 can be selected as a dose of WITHASOL for immunostimulatory activity and anti-tumor potentiation activity.

Although doses R-1, R-2 and R-4 did not appear to exhibit statistically significant protective activity against myelosuppression in these particular studies, this may be due to the greater severity of tumor-induced leukocytosis (P=0.0003) than in the earlier study (P=0.04) and the myelosuppressive effect of CP was not highly evident in group IV animals. However, an almost linear dose activity relationship with peak activity to R-3 was observed. The aforementioned animal studies demonstrate that WITHASOL provides both short and long term benefits to animals having immunopathological disorders by protecting the animals from CP induced myelo and immunosuppression as evidenced by increased white cell total counts and antibody titres.

1. Withania somnifera root extract:

Description: Brown colored, viscous, homogenous free flowing suspension with characteristic odor and bitter taste.

LOD at 900° C. under 15 mm Hg for 4 hrs.: 35%

Wt./ml: 1.3377 g

TLC pattern: Matching with the Working Standard.

Assay of actives:

Withanolide D: 0.111% (as is basis)

Withaferin A: 0.134% (as is basis)

2. Withasol (prepared from Withania somnifera root extract of above specifications)

The extract product was subjected to vacuum to remove traces of residual solvent. The composition was the same as confirmed by superimposition of TLC and HPLC profiles on the previous product.

Standardization of the extract:

Appearance: Dark red, highly viscous, sticky liquid.

Yield: 80.0% (HPR-approx.7:1)

LOD: 35.0%

pH of 5% soln.: 5.4

Limit test for Heavy metals (IP 1996): Passes (20 ppm)

Limit test for Arsenic (IP 1996): Passes (10 ppm)

Glycowithanolides (In house assay): 1.017%

Protein content (By Kjeldhal method, IP 1996): 47.8%

Alkaloids content (by Dragendorf test): Absent

Total Ash content (IP 1996 method): 7.26%

Acid insoluble ash (IP 1996 method): 1.603%

What is claimed is:

1. A method for obtaining a composition having immune stimulating activity or anti-tumor activity from Withania Somnifera comprising:
(a) contacting Withania Somnifera plant or plant part with a first medium polar solvent to produce a particulate suspension;
(b) clarifying the particulate suspension to produce a clarified first solution and a first residue;
(c) evaporating the solvent from the first clarified solution to produce a fraction, denoted fraction A;
(d) resuspending the first residue in a second polar solvent thereby producing a second solution and a second residue;
(e) clarifying the second solution to produce a second clarified solution;
(f) evaporating the second polar solvent from the second clarified solution to produce a fraction, denoted fraction B;
(g) resuspending the second residue in a third solvent more polar than the second polar solvent thereby producing a third solution and a third residue;
(h) clarifying the third solution to produce a third clarified solution;
(i) evaporating the third solvent from the third clarified solution to produce a fraction, denoted fraction C;
(j) combining fractions A, B and C to produce an extract;
(k) resuspending the extract in a solution to produce a fourth alkaline solution; and
(l) fractionating the fourth solution with a non polar solvent and removing the solvent to produce a composition having immune stimulating activity or anti-tumor activity.

2. The method of claim 1, wherein fractions A, B and C are combined in approximately equal proportions by mass.

3. The method of claim 1, wherein fractions A, B and C are combined in unequal proportions by mass.

4. The method of claim 1, wherein the first residue is resuspended in a solvent having about 50% ethanol or about 40 to 60% isopropyl alcohol.

5. The method of claim 1, wherein the second residue is resuspended in water.

6. The method of claim 1, wherein step 1) removes one or more alkaloids.

7. The method of claim 1, wherein step 1) removes one or more withanolides.

8. The method of claim 1, wherein step 1) comprises fractionating the extract with methylene chloride, diethyl ether or chloroform.

9. The method of claim 1, wherein the plant part comprises a root.

10. The method of claim 1, wherein the first medium polar solvent comprises acetone, tetrahydrofuran or ethylacetate.

11. The method of claim 1, wherein the second solvent comprises a mixture of water and isopropyl alcohol (IPA).

12. The method of claim 1, wherein the third solvent comprises water.

13. The method of claim 1, wherein the first or second solvent comprises an alcoholic organic solvent.

14. The method of claim 1, wherein step a) comprises soaking the plant or plant part in the first solvent for at least about 2 hours.

15. A composition having immune stimulating activity or anti-tumor activity produced by the method of claim 1.

16. The composition of claim 15, wherein the composition is characterized as having a TLC profile the same as a profile set forth in FIGS. 1A, 2A or 3A, the profile obtained with a hexane:methylene chloride:methanol mobile phase in about a 20:30:2 ratio.

17. The composition of claim 15, wherein the composition is characterized as having an HPLC profile substantially the same as a profile set forth in FIGS. 1B, 2B or 3B, said profile obtained using a reverse-phase C-18 column at a flow rate of about 1.2 ml/min with a mobile phase of methanol:water in a ratio of about 60:40.

18. A composition having immune stimulating activity or anti-tumor activity, said composition comprising any one of the molecules in peaks 1 to 5 or 7 to 9 set forth in FIGS. 1A, 2A or 3A, or a combination of two or more molecules in said peaks.

19. A composition obtained from Withania Somnifera characterized as:
(a) having immune stimulating activity or anti-tumor activity;
(b) soluble in water;
(c) substantially free of alkaloids; and
(d) having at least one glycowithanolide.

20. The composition of claim 19, further characterized as having a TLC profile substantially the same as a profile set forth in FIGS. 1A, 2A or 3A, the profile obtained with a hexane:methylene chloride:methanol mobile phase in about a 20:30:2 ratio.

21. The composition of claim 19, further characterized as having an HPLC profile substantially the same as a profile set forth in FIGS. 1B, 2B or 3B, said profile obtained using a reverse-phase C-18 column at a flow rate of about 1.2 ml/min with a mobile phase of methanol:water in a ratio of about 60:40.

22. The composition of claim 19, further characterized as substantially free of withanolides.

23. The composition of claim 19, further characterized as having a glycowithanolide content from about 0.5 to 1.6% by weight.

24. The composition of claim 19, wherein the glycowithanolide comprises sitoindoside IX.

25. The composition of claim 19, wherein the glycowithanolide comprises sitoindoside X.

26. The composition of claim 19, wherein the glycowithanolide comprises a mixture of sitoindoside IX and sitoindoside X.

27. The composition of claim 19, wherein the glycowithanolide comprises a mixture of sitoindoside IX, sitoindoside X and one or more glycowithanolides distinct from sitoindoside IX and sitoindoside X.

28. The composition of claim 19, wherein the glycowithanolide comprises a mixture of sitoindoside IX, sitoindoside X and two or more glycowithanolides distinct from sitoindoside IX and sitoindoside X.

29. The composition of claim 19, wherein the glycowithanolide comprises a mixture of sitoindoside IX, sitoindoside X and three or more glycowithanolides distinct from sitoindoside IX and sitoindoside X.

30. The composition of claim 19, further characterized as having one or more of the following by mass:
(a) about 35-75% protein content;
(b) about 0.5 to about 5% glycowithanolide(s);
(c) about 3 to about 10% ash; and
(d) about 30 to about 60% carbohydrate.

31. The composition of claim 19, wherein administering about 50 mg/kg subject mass of the composition to a Balb-c mouse increases by about 20% or more the number of white blood cells in the Balb-c mouse.

32. The composition of claim 31, wherein the subject has less than normal numbers of white blood cells.

33. The composition of claim 19, wherein administering about 100 mg/kg subject mass of the composition to a Balb-c mouse increases by about 20% or more the number of white blood cells in the Balb-c mouse.

34. The composition of claim 33, wherein the subject has less than normal numbers of white blood cells.

35. A pharmaceutical formulation comprising the composition of claim 17, and a pharmaceutically acceptable carrier.

36. The formulation of claim 35, further comprising a drug.

37. The formulation of claim 35, wherein the drug increases white blood cell numbers in a subject.

38. The formulation of claim 35, wherein the drug has immunosuppressing activity in a subject.

39. The formulation of claim 35, wherein the drug inhibits cell cycle progression.

40. The formulation of claim 35, wherein the drug inhibits cell proliferation.

41. The formulation of claim 35, wherein the drug comprises an anti-tumor drug.

42. The formulation of claim 41, wherein the anti-tumor drug inhibits nucleic acid or protein synthesis.

43. The formulation of claim 38, wherein the drug comprises a steroid glycoside.

44. The formulation of claim 35, wherein the drug comprises an alkylating agent, an anti-metabolite, a plant alkaloid, a plant extract, an antibiotic, a nitrosourea, a hormone, a nucleoside analogue, or a nucleotide analogue.

45. The formulation of claim 35, wherein the drug is selected from cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-AZC, taxol, vinblastine, vincristine, doxorubicin, bleomycin, actinomycin D, mithramycin, mitomycin C, carmustine, lomustine, semustine, streptozotocin, hydroxyurea, cisplatin, mitotane, procarbazine, dacarbazine or dibromomannitol.

46. The formulation of claim 35, wherein the excipient is suitable for injection or infusion.

47. The formulation of claim 35, wherein the formulation comprises a pill, granules, crystals, a capsule, a syrup, a suspension, an elixir or an injectable.

48. A kit comprising the pharmaceutical formulation of claim 35, and instructions for use in stimulating an immune response or in potentiating anti-cell proliferative activity of an anti-cell proliferative therapy

49. A method for increasing the number of white blood cells in a subject comprising administering to a subject an amount of the composition of claim 17 effective to increase the number of white blood cells in the subject.

50. The method of claim 49, wherein the white blood cells are selected from monocytes, macrophages, natural killer cells, dendritic cells, granulocytes, basophils and eosinophils.

51. The method of claim 49, wherein the subject has less than normal numbers of white blood cells.

52. The method of claim 49, wherein the subject has been, is currently undergoing or will be undergoing an immunosuppressive therapy.

53. The method of claim 49, wherein the subject has been, is currently undergoing or will be undergoing a cancer therapy.

54. The method of claim 53, wherein the cancer therapy comprises administration of radiation or a radioisotope.

55. The method of claim 53, wherein the subject has asthma, rheumatoid arthritis, or psoriasis.

56. A method for reducing immunosuppression in a subject comprising administering to an immunosuppressed subject, or a subject at risk of immunosuppression, an amount of the composition of claim 19 effective to reduce immunosuppression in the subject.

57. The method of claim 49 or 56, wherein the subject is treated prohylactically.

58. The method of claim 49 or 56, wherein the amount administered comprises a dose of about 10 to 50, 50 to 100, or 100 to 200 mg composition/kg subject mass.

59. The method of claim 49 or 56, wherein the composition is administered in multiple doses.

60. The method of claim 49 or 56, wherein the composition is administered via injection, gradual perfusion or intubation.

61. The method of claim 49 or 56, wherein the composition is administered orally.

62. The method of claim 49 or 56, wherein the composition is administered prior to, contemporaneously with, or after administering a drug.

63. The method of claim 62, wherein the drug stimulates or suppresses an immune response.

64. A method for increasing activity of an anti-tumor drug comprising administering to a subject treated with an anti-tumor drug the composition of claim 19 prior to, contemporaneously with or after administering the anti-tumor drug to the subject.

65. The method of claim 64, wherein the anti-tumor drug comprises a radioisotope, an alkylating agent, an anti-metabolite, a plant alkaloid, a plant extract, an antibiotic, a nitrosourea, a hormone, a nucleoside analogue, or a nucleotide analogue.

66. The method of claim 64, wherein the amount administered comprises a dose of about 10 to 50, 50 to 100, or 100 to 200 mg composition/kg subject mass.

67. The method of claim 64, wherein the subject is at risk of, presently has or previously had cancer.

68. The method of claim 67, wherein the cancer comprises a solid or liquid tumor.

69. The method of claim 68, wherein the cancer comprises a breast, brain, head or neck, eye, nasopharynx, lung, liver, pancreas, kidney, esophagus, stomach, small or large intestine, bladder, rectal, prostate, testicular, ovarian, uterine, bone, muscle or skin tumor.

70. The method of claim 68, wherein the solid tumor comprises a fibrosarcoma, lymphosarcoma, liposarcoma or osteosarcoma.

71. The method of claim 68, wherein the liquid tumor comprises a lymphoma, leukemia or myeloma.

72. The method of claim 64, wherein the composition is administered at intermittent frequencies or variable dosages.
